SEQUENCE LISTING (5 sequences)

SEQ ID NO: 1 - protein, 498 aa, Dichomitus squalens
AIGPVADMTVTDDNISPDGYSRAAVLVNGVFPSPLITGNKGDNFQLNVIDNLTNDTMLTATTIHWHGFFQKGTNYADGPAFVNQCPISKGNSFLYDFTATDQAGTFWYHSHLSTQYCDGLRGAFVVYDPNDPHASLYDVDDESTVITLADWYHTAARLGPRFPLGADTVLINGIGRFTGGDAVDLAVISVTQGKRYRFRLVSISCDPNFTFSIDNHNLTIIEVDAVNHQPLTVDEIQIFAGQRYSFVLTADQAIDNYWIRALPQIGTVSFDGGVNSAILRYDGAAAIEPTTNQTGGSNPLVEANLVPLDNLAAPGSAVVGGVDWAHSFDFSFNGTEFFIDGTPFVSPSVPVLLQILSGAQSASDLLPSGSVFSLPSNSTIELSFPLTATNAPGGPHPFHLHGHTFYVVQSAGQTEPNYVNPPQRDVVSTGTAGDNVTIRFTTNNPGPWFLHCHIDFHLEAGFAVVLAEDIPDVSSANTPTTAWENLCPTYSQVYPSGL

SEQ ID NO: 2 - protein, 498 aa, Phlebia radiata
GIGPVTDLRISNAVVSPDGFSRDAVVVQGQFPSPLIKGNKGDMFKLNVINQLQDTALNTSTSIHWHGLFQHGTNWADGPAFVTQCPIVTGDSFVYDFTVPDQAGTFWYHSHLALQYCDGLRGPLVVYDPHDPYAHLYDVDDESTVITLAEWYHTAADNLRPPEEANSTLINGLGRYAGGPASPLSVITVEHGKRYRFRLVSMACDPSYNFTIDGHNMTVIEVDGVNHLPLTVDKIQIFVAQRYSFILDANQPIGNYWIHAIPNGQSQLVGVANGINSAILRYVGAPNKEPTTQTPPSVAPLQEVNLHPLVNAAAPGKPFPGGADQVIPFNLSLLGANFLINNATFSPPTVPVLLQILSGAKTPQQLLPPGSIYSLKRNSVIEIVIPPGTAPGGPHPFHLHGHTFSVVRSAGSSVYNFKNPVQRDVVSIGTGANDSVTIRFVTDNPGPWFIHCHIDFHLNAGLAAVMAEDIPDIKLANPVPAEWEQLCPIYDQKVLHEH

SEQ ID NO: 3 - protein, 499 aa, synthetic construct
AIGPSANLVVTDAPIAPDGFLRDAIVVNGVFPSPLITGKKGDRFQLNVVDKLANHTMLKSTSIHWHGFFQAGTNWADGPAFVNQCPIASGHSFLYDFHVPDQAGTFWYHSHLSTQYCDGLRGPFVVYDPKDPHASRYDVDNESTVITLTDWYHTAARLGPKAPLGADATLINGLGRSTSTPTAALAVINVQHGKRYRFRLVSISCDPNYTFSIDGHNMTIIEVDGINSQPLLVDSIQIFPAQRYSFVLNANQTVGNYWVRANPNFGTVGFAGGINSAILRYEGAPVAEPNTTQTPSVIPLIETNLHPLARMPVPGSPTPGGVDKALNLAFNFNGTNFFINNASFTPPTVPVLLQILSGAQTAQDLLPAGSVYPLPAHSSIEITMPATALAPGAPHPFHLHGHAFAVVRSAGSTTYNYNDPIFRDVVNTGTPAAGDNVTIRFQTDNPGPWFLHCHIDFHLDAGFTIVFAEDVADVKAANPVPKAWSDLCPIYDGLGEADQ

SEQ ID NO: 4 - protein, 505 aa, Coprinopsis cinerea
QILGPTSTMTVSNIDASPDGFNRPVVAVNGQHPGPLVRANKGDNFRINVVNDLNDPTMLRQTSVHWHGVFQHGTAWADGPDGVTQCPIAQNGESFEYRFNAGNEAGTFWYHSHFGTQYCDGLRGPLVIYDPNDPHRNLYDVDNADTVITLVDWYHLQAPSIEGPALSDATLINGKGRRPGGPETDIAIVNVQRNRRYRFRLVSMSCDPNYKFSIDGHKLTVIEADGQLTEPLMVDEIQIFAGQRYSFVLSANRPVGNYWIRAIPNVGSNNLPNFSSGGINSAILRYAGAPNANPTSTPVTNPVALHESNLHALLNPGAPGGSGPADENIVLQMGLGPAGFEINGVTWANPDSPVMVQIMNGVPPADIVPSGAIHTLPRNRVVEVSIPGFELAGPHPFHLHGHAFSVVRSAGSSTYNYENPVRRDVVDVGGASDNVTIRFTTDNPGPWFFHCHIEFHLVLGLAMVFMEAPSDIPSTSPPPPSWSELCPKFESLPASATSIQIVPTP

SEQ ID NO: 5 - protein, 497 aa, synthetic construct
AIGPVTDLTITDVSISPDGFERAAVLAGGTFPGPLITGNIGDNFQINVVNQLSNETMLESTTIHWHGLFQKGTAWADGPAFVSQCPIATGNSFLYDFSVPDQAGTFWYHSHLATQYCDGLRGPLVVYDPNDAYLSDYDVDDDTTVITLADWYHTAATLGPRFPTQADATLINGLGRYSGGPESNLTVITVESGKRYRFRLVSISCNENYVFSIDDHEMIVIEADGVETQPVTVDSIQIFAAQRYSFILNATEAVDNYWIRATPDLGTTGFTDGINSAILRYVGANDTDPTTASNTTNLLSELDLHPLTNAAAPGAASQDPGSVDVSMNLELGFDGTNFTINGATFVPPTVPVLLQILSGAQSATDLLPSGSVFTLPSNSTVQVTLAAGTVAGGPHPFHLHGHTFSVVRSAGSDTYNYDNPVQRDTVSIGADGDNVTIRFDTNNPGPWFLHCHIDWHLNAGFAIVFAEDTADIASANPVPDSWSDLCPTYDALPSDEL

(19) United States
(12) Patent Application Publication
(10) Pub. No.: US 2026/0275050 A1
(43) Pub. Date: Sep. 17, 2026

(54) METHOD FOR PRODUCING LIGNIN-HEMICELLULOSE HYBRID NANOPARTICLES OR LIGNIN NANOPARTICLES

(71) Applicant: Helsingin yliopisto, Helsingin yliopisto (FI)

(72) Inventors: Kirsi S. MIKKONEN, Helsingin yliopisto (FI); Maarit LAHTINEN, Helsingin yliopisto (FI); Kristiina HILDEN, Helsingin yliopisto (FI); Patrícia FIGUEIREDO, Helsingin yliopisto (FI); Danila MORAIS DE CARVALHO, Helsingin yliopisto (FI); Mina ZARE, Helsingin yliopisto (FI)

(21) Appl. No.: 19/165,867

(22) PCT Filed: Mar. 15, 2024
(86) PCT No.: PCT/FI2024/050120

§ 371 Date: Sep. 16, 2025

(30) Foreign Application Priority Data
Mar. 17, 2023 (FI) 20235314

(51) Int. Cl. C08H 8/00

(52) U.S. Cl. CPC C08H 8/00

(57) ABSTRACT

A method for producing lignin-hemicellulose hybrid nanoparticles, each of the lignin-hemicellulose hybrid nanoparticles comprising a lignin nanoparticle, is disclosed. The method may comprise adding hemicellulose to the surfaces of the lignin nanoparticles, thereby covering the lignin nanoparticles with hemicellulose.

### TECHNICAL FIELD

The present disclosure relates to a method for producing lignin-hemicellulose hybrid nanoparticles and lignin nanoparticles; to lignin-hemicellulose hybrid nanoparticles and lignin nanoparticles; to compositions or products comprising the same; and to a laccase enzyme.

### BACKGROUND

Lignin is one of the most prevalent renewable resources based on aromatic units and most abundant polymers on Earth and isolated from lignocellulosic biomass. Lignin is a complex polyphenolic macromolecule that may be derived from different sources of plant biomass, such as hardwood, softwood and straw or grass. It is composed of three basic monomeric units—syringyl (S), guaiacyl (G), and p-hydroxyphenyl (H)—that vary according to the source and extraction method.

There is interest in the use of lignin for advanced applications, mainly due to its remarkable potential and unique properties, including UV-blocking, antimicrobial and antioxidant abilities, as well as biodegradability and biocompatibility. Nevertheless, valorization of lignin has been challenged by its complex and heterogeneous molecular structure, the composition of which can vary according to the extraction method and source of lignin.

The modification and functionalization of lignin may increase the application of such systems by providing new functionalities and stabilizing against organic solvents. However, these modifications are conventionally performed using harsh and energy-demanding methods.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A method for producing lignin-hemicellulose hybrid nanoparticles, each of the lignin-hemicellulose hybrid nanoparticles comprising a lignin nanoparticle, is disclosed. The method may comprise adding hemicellulose to surfaces of the lignin nanoparticles, thereby covering the lignin nanoparticles with hemicellulose.

Lignin-hemicellulose hybrid nanoparticles are disclosed. Each of the lignin-hemicellulose hybrid nanoparticles may comprise a lignin nanoparticle and hemicellulose covering the lignin nanoparticle.

A method for producing and/or modifying lignin nanoparticles is disclosed. The method may comprise contacting a laccase enzyme with the lignin nanoparticles, thereby modifying one or more properties of the lignin nanoparticles.

Lignin nanoparticles are also disclosed.

### BRIEF DESCRIPTION OF THE DRAWINGS

The accompanying drawings, which are included to provide a further understanding of the invention and constitute a part of this specification, illustrate embodiments and together with the description help to explain the principles of the embodiments. In the drawings:

FIG. 1 shows the size of the lignin nanoparticles (LNPs) measured by dynamic light scattering after incubation of LNPs with the different laccases (1000 nKat/g of lignin) and hemicelluloses (0.5-5 mg/mL), at pH 5 and RT for 24 h. Error bars represent the mean±s.d. (n≥3);

FIG. 2 shows surface charge of LNPs, given by the ζ-potential, measured by dynamic light scattering after incubation of LNPs with the different laccases (1000 nKat/g of lignin) and hemicelluloses (0.5-5 mg/mL), at pH 5 and RT for 24 h. Error bars represent the mean±s.d. (n≥3);

FIG. 3 shows the stability of LNPs suspensions in 25 mM citric acid pH 3 by dynamic light scattering in terms of their size, after incubation with the different laccases (1000 nKat/g of lignin) and hemicelluloses (0.5-5 mg/mL), at pH 5 and RT for 24 h. Error bars represent the mean±s.d. (n≥3);

FIG. 4 shows the stability of LNPs suspensions in 25 mM citric acid pH 3 by dynamic light scattering in terms of their surface charge, after incubation with the different laccases (1000 nKat/g of lignin) and hemicelluloses (0.5-5 mg/mL), at pH 5 and RT for 24 h. Error bars represent the mean±s.d. (n≥3); and

FIG. 5 illustrates colloidal stability of LNPs up to 30 days storage, in terms of variation in the transmitted light through the LNP suspensions in MilliQ-water, evaluated using Turbiscan. The LNPs were previously incubated with the different laccases (1000 nKat/g of lignin) and hemicelluloses (5 mg/mL), at pH 5 and RT for 24 h. Error bars represent the mean±s.d. (n=2) For the last time point, the samples were mixed in order to redispersed the LNPs.

FIG. 6 illustrates Py-GCMS analysis of a) LB-LNPS (softwood); b) PB-LNPs (wheat straw/Sarkanda grass), and c) BB-LNPS (hardwood) suspensions after incubation with the different laccases at 1000 nKat g⁻¹ (pH 5) for 24 h at RT;

FIG. 7 shows the quantification of the absorbance at 515 nm of a,d) LB-LNPs (softwood); b,e) PB-LNPs (wheat straw/Sarkanda grass), and c,f) BB-LNPs (hardwood) suspensions after incubation with the different laccases (100-1000 nKat g⁻¹), at pH 5 and RT, for a-c) 1 h and d-f) 24 h, using UV/Vis spectroscopy;

FIG. 8 shows the quantification of the phenolic content of a,d) LB-LNPs (softwood); b,e) PB-LNPs (wheat straw/Sarkanda grass), and c,f) BB-LNPs (hardwood) suspensions after incubation with the different laccases (100-1000 nKat g⁻¹), at pH 5 and RT, for a-c) 1 h and d-f) 24 h, using UV/Vis spectroscopy (Folin-Ciocalteu reagent) with vanillin as standard. Error bars represent the mean±s.d. (n=3);

FIG. 9 illustrates the characterization of a,d) LB-LNPs (softwood); b,e) PB-LNPs (wheat straw/Sarkanda grass), and c,f) BB-LNPs (hardwood) by dynamic light scattering in terms of their size, after incubation with the different laccases (100-1000 nKat g⁻¹), at pH 5 and RT, after a-c) 1 h and d-f) 24 h. Error bars represent the mean±s.d. (n≥3);

FIG. 10 illustrates the characterization of a,d) LB-LNPs (softwood); b,e) PB-LNPs (wheat straw/Sarkanda grass), and c,f) BB-LNPs (hardwood) by dynamic light scattering in terms of ζ-potential after incubation with the different laccases (100-1000 nKat g⁻¹), at pH 5 and RT, after a-c) 1 h and d-f) 24 h. Error bars represent the mean±s.d. (n≥3);

FIG. 11 shows the size of the LNPs measured by dynamic light scattering after incubation of LNPs with the different laccases (1000 nKat/g of lignin) and other coatings, such as tannic acid and pure BLN GGM hemicellulose (1 and 2.5 mg/mL), at pH 5 and RT for 24 h. Error bars represent the mean±s.d. (n≥3);

FIG. 12 shows the ζ-potential of the LNPs measured by dynamic light scattering after incubation of LNPs with the different laccases (1000 nKat/g of lignin) and other coatings, such as tannic acid and pure BLN GGM hemicellulose (1 and 2.5 mg/mL), at pH 5 and RT for 24 h. Error bars represent the mean±s.d. (n≥3);

FIG. 13 shows Py-GCMS analysis of LNPs after their incubation with the different laccases (1000 nKat/g of lignin) and hemicelluloses (2.5 mg/mL), at pH 5 and RT for 24 h;

FIG. 14 illustrates the monosaccharide composition of LNPs functionalized with hemicelluloses (2.5 mg/mL) by spontaneous absorption (no laccase), and treated with DsLcc4 (1000 nKat/g of lignin), at pH 5 and RT for 24 h, determined using acid methanolysis analysis followed by the gas chromatography detection; and

FIG. 15 shows contact angle measurements representing the wettability (hydrophobicity and hydrophilicity) of LB-LNPs, BB-LNPs, and functionalized INPS by hemicellulose, with and without laccase (DsLcc4) treatment.

### DETAILED DESCRIPTION

According to a first aspect, a method for producing lignin-hemicellulose hybrid nanoparticles is provided. Each of the lignin-hemicellulose hybrid nanoparticles may comprise a lignin nanoparticle. Each of the lignin nanoparticles may have a surface. The method may comprise adding hemicellulose to the surfaces of the lignin nanoparticles, thereby covering the lignin nanoparticles with the hemicellulose and optionally covalently crosslinking the hemicellulose to the lignin nanoparticles.

According to the first aspect, lignin-hemicellulose hybrid nanoparticles are also provided. Each of the lignin-hemicellulose hybrid nanoparticles may comprise a lignin nanoparticle and hemicellulose covering the lignin nanoparticle. Each of the lignin nanoparticles may have a surface. The hemicellulose may thus cover the surface of the lignin nanoparticle(s). The hemicellulose may optionally be covalently crosslinked to the lignin nanoparticle.

Any embodiments and disclosure set out below may be considered to relate to both the first and the second aspect described in this specification, unless otherwise mentioned.

The method may comprise adding hemicellulose to the surfaces of the lignin nanoparticles, thereby covering the lignin nanoparticles with the hemicellulose. The hemicellulose may be adsorbed to the lignin nanoparticles, i.e. to the surfaces of the lignin nanoparticles. The lignin-hemicellulose hybrid nanoparticles may thus be considered to be coated by the hemicellulose, i.e. to comprise a hemicellulose coating.

The lignin may contain a minor amount of hemicellulose, depending e.g. on the source and production process of the lignin. Likewise, the hemicellulose may contain a minor amount of lignin, depending e.g. on the source and production process of the hemicellulose. Thus the hemicellulose may be concentrated at the surface of the lignin-hemicellulose hybrid nanoparticles.

The method may further comprise covalently crosslinking the hemicellulose to the lignin nanoparticles.

The hemicellulose may comprise or be xylan, glucuronoxylan, arabinoxylan, glucomannan, xyloglucan. galactoglucomannan, and/or any mixture or combination thereof. The hemicellulose may be obtainable or obtained from softwood and/or hardwood. The hemicellulose may be obtainable through hot water extraction of wood (e.g. softwood and/or hardwood). The hemicellulose may comprise or be e.g. spray dried and/or ethanol-precipitated galactoglucomannan, for example from softwood, and/or e.g. spray dried and/or ethanol-precipitated glucuronoxylan, for example from hardwood. The chemical composition of the hemicellulose may depend on the source from which it has been obtained.

The hemicelluloses used for the preparation of lignin-hemicellulose hybrid nanoparticles are extracted from different sources, such as spruce galactoglucomannan (GGM) and birch glucuronoxylans (GX), and then spray dried (sd) or ethanol precipitated (ep).

The hemicellulose may thus comprise or be galactoglucomannan (e.g. spruce galactoglucomannan), glucuronoxylan (e.g. birch glucuronoxylan), or any mixture or combination thereof. The hemicellulose may be spray dried or ethanol precipitated.

In the context of this specification, the term “hemicellulose” may also be understood as referring to any mixture or combination of two or more types of hemicelluloses, or to a composition comprising hemicellulose or any mixture or combination of two or more types of hemicelluloses.

The hemicellulose may be provided or obtained/obtainable as a composition comprising the hemicellulose. The composition may comprise e.g. at least 60%, or 60-98%, or 60-100% (w/w) of hemicellulose based on the dry weight of the composition. Such a composition may also comprise also other components, for example a small amount of lignin residues. The lignin may at least partially be covalently bound with the hemicellulose via lignin-carbohydrate complexes (LCCs). In such embodiments, the composition comprising the hemicellulose may be added to the surfaces of the lignin nanoparticles.

The hemicellulose may be provided e.g. as a composition comprising the hemicellulose dissolved in a suitable solvent such as an aqueous solution, for example an aqueous solution comprising 25 mM citric acid at pH 5.

With the method according to the first aspect, may be possible to tailor surface or other properties of the lignin-hemicellulose hybrid nanoparticles e.g. to a particular application or end use.

It may, additionally or alternatively, be possible to obtain lignin-hemicellulose hybrid nanoparticles that are relatively homogeneous and have a desired particle size distribution.

The lignin-hemicellulose hybrid nanoparticles may be more stable than lignin nanoparticles that do not contain hemicellulose. For example, they may have a reduced tendency to aggregate. The hemicellulose may stabilize the surface of the lignin-hemicellulose hybrid nanoparticles.

It may also be possible to use technical lignins in the method. However, some technical lignins may not be readily soluble e.g. in acidic aqueous solutions, which may render them less suited for the method.

The method may further comprise contacting a laccase enzyme with the hemicellulose and the lignin nanoparticles and/or the lignin-hemicellulose hybrid nanoparticles. Not to be bound by theory, the laccase enzyme may thereby covalently crosslink the hemicellulose to the lignin nanoparticles. The laccase enzyme treatment may improve the stability of the lignin-hemicellulose hybrid nanoparticles. However, the hemicellulose may at least in some embodiments be added to the lignin nanoparticles without a laccase treatment, thereby covering the lignin nanoparticles with the hemicellulose. In such embodiments, the hemicellulose may be adsorbed to the surfaces of the lignin nanoparticles.

The laccase enzyme may first be contacted with the lignin nanoparticles, and subsequently the hemicellulose may be added to the surfaces of the lignin nanoparticles, thereby covering the lignin nanoparticles with the hemicellulose.

In some embodiments, the laccase enzyme may be contacted with the lignin nanoparticles and the hemicellulose may be added to the surfaces of the lignin nanoparticles simultaneously, thereby covering the lignin nanoparticles with the hemicellulose.

In some embodiments, the hemicellulose may first be added to the surfaces of the lignin nanoparticles, thereby covering the lignin nanoparticles with the hemicellulose, and then the laccase enzyme may be contacted with the lignin-hemicellulose hybrid nanoparticles. However, the hemicellulose adsorbed to and covering the surfaces may then prevent, at least to some extent, reactions catalysed by the laccase enzyme.

The conditions of the laccase treatment may be selected depending e.g. on the exact laccase enzyme used and depending on the conditions required to keep the lignin nanoparticles and/or the lignin-hemicellulose hybrid nanoparticles stable. The laccase enzyme may be contacted with the hemicellulose and the lignin nanoparticles and/or with the lignin-hemicellulose hybrid nanoparticles e.g. at a pH in the range of 4 to 7, or 5 to 6. The laccase treatment may be performed e.g. in an aqueous solution comprising 25 mM citric acid at a pH of 5, for example for a period of 1 to 24 hours. The laccase enzyme may be used e.g. as an amount of 100-1000 nKat/g based on the total dry weight of the lignin in the lignin nanoparticles. The laccase enzyme may be contacted with the hemicellulose and the lignin nanoparticles and/or with the lignin-hemicellulose hybrid nanoparticles e.g. at room temperature. The term “room temperature” may be considered to refer to a temperature in the range of 20 to 25° C. After the laccase treatment, the resulting lignin nanoparticles and/or the lignin-hemicellulose hybrid nanoparticles may be purified by centrifugation and resuspended e.g. in water.

In the context of this specification, the term “water” may be understood as referring to pure or ultrapure water, for example to Milli-Q purified water, or, in some embodiments, e.g. to tap water.

In some embodiments, the laccase enzyme may be such that is not capable of oxidizing hemicellulose and/or proteins. In other words, the laccase enzyme may not be capable of oxidizing the carbohydrate part of the hemicellulose. It may not be desirable for the laccase enzyme to chemically alter the structure of the hemicellulose and/or proteins. However, hemicellulose may be provided and/or obtainable as a composition comprising the hemicellulose but also other components, for example a small amount of lignin residues. In some compositions comprising hemicellulose, the lignin content could be as high as 2-35% (w/w). In such embodiments, the laccase enzyme may catalyze crosslinking of the composition comprising the hemicellulose and/or the hemicellulose to the lignin nanoparticles (at least to some extent). In such cross-linking, the lignin may play a role.

The lignin-hemicellulose hybrid nanoparticles may be obtainable or obtained by the method according to one or more embodiments described in this specification.

The hemicellulose may increase the ζ-potential of the lignin-hemicellulose hybrid nanoparticles as compared to lignin nanoparticles without hemicellulose. I.e. the ζ-potential of the lignin-hemicellulose hybrid nanoparticles may change towards a higher value, i.e. a less negative value (towards zero).

The ζ-potential of the lignin-hemicellulose hybrid nanoparticles in water may be −42 mV or higher. In some embodiments, the ζ-potential of the lignin-hemicellulose hybrid nanoparticles in water may be −42 mV or lower, or in the range of −45-−42 mV, or in the range of −42-0 mV. The ζ-potential of the lignin-hemicellulose may be measured e.g. by dynamic light scattering, for example as set out in the Examples. For the measurement, the lignin-hemicellulose hybrid nanoparticles or lignin nanoparticles may be suspended in water (e.g. MilliQ-water) e.g. at a concentration of 500 μg/mL. The measurement may be performed using a Malvern Zetasizer Nano ZS instrument.

The hemicellulose may reduce the tendency of the lignin-hemicellulose hybrid nanoparticles to aggregate, such that the average or median particle size of the lignin-hemicellulose hybrid nanoparticles and/or their polydispersity index does not increase, for example during storage.

In the context of this specification, the term “lignin-hemicellulose hybrid nanoparticle” should not be understood as necessarily being limited to any particular particle size. It may be understood as referring to lignin-hemicellulose hybrid nanoparticles having e.g. a particle size, for example a median or average particle size, in the range of 1-1000 nm.

The average or median particle size of the lignin-hemicellulose hybrid nanoparticles may be in the range of 100-500 nm, or in the range of 100-200 nm. The average or median particle size may be measured e.g. by dynamic light scattering, for example as set out in the Examples. For the measurement, the lignin-hemicellulose hybrid nanoparticles or lignin nanoparticles may be suspended in water (e.g. MilliQ-water) e.g. at a concentration of 500 μg/mL. The measurement may be performed using a Malvern Zetasizer Nano ZS instrument.

The polydispersity index (PDI) of the lignin-hemicellulose hybrid nanoparticles may be e.g. 0.15 or lower. The PDI of the lignin-hemicellulose hybrid nanoparticles may be e.g. in the range of 0.01-0.15. The PDI may be measured e.g. by dynamic light scattering, for example as set out in the Examples. For the measurement, the lignin-hemicellulose hybrid nanoparticles or lignin nanoparticles may be suspended in water (e.g. MilliQ-water) e.g. at a concentration of 500 μg/mL. The measurement may be performed using a Malvern Zetasizer Nano ZS instrument.

The presence of the hemicellulose may increase the stability (e.g. colloidal stability) of the lignin-hemicellulose hybrid nanoparticles. The lignin-hemicellulose hybrid nanoparticles may be stable in water or an aqueous solution for at least 4 weeks. The pH of the aqueous solution may be about 7. The lignin-hemicellulose hybrid nanoparticles may be stable in the water or the aqueous solution for at least 4 weeks in storage, for example at a temperature of about 4° C. The lignin-hemicellulose hybrid nanoparticles may be stable in the water or the aqueous solution longer, for at least 7 weeks, or at least one month, or at least 2 months, or at least 3 months.

The lignin-hemicellulose hybrid nanoparticles may be stable in an aqueous solution having a pH of about 3 for at least 2 hours, or at least one day, or at least 7 days, for example at a temperature of about 4° C. or at room temperature. The aqueous solution having the pH of about 3 may be e.g. a 25 mM citric acid solution having a pH of about 3. The stability of the lignin-hemicellulose hybrid nanoparticles at an acidic pH, for example at a pH of about 3, may be relevant e.g. for lignin-hemicellulose hybrid nanoparticles that may be delivered to the gastrointestinal tract.

In some embodiments, the lignin-hemicellulose hybrid nanoparticles may be considered to be stable in the aqueous solution, when their polydispersity index remains at e.g. 0.15 or lower or in the range of 0.01-0.15 in the water or the aqueous solution for the at least 4 weeks, or the at least 2 hours (or other time period as set out in this specification).

In some embodiments, the lignin-hemicellulose hybrid nanoparticles may be considered to be stable in the water or the aqueous solution, when the average or median particle size of the lignin-hemicellulose hybrid nanoparticles remains in the range of 100-500 nm, or in the range of 100-200 mm in the aqueous solution for the at least 4 weeks, or the at least 2 hours (or other time period as set out in this specification).

The lignin may be technical lignin. In the context of this specification, the term “technical lignin” may be understood as referring to lignin obtainable as isolated from biomass using a technical process, such as a pulping process. Such lignin typically has a different chemical structure from natural lignin.

The lignin may be e.g. lignin obtained from softwood, hardwood, and/or a grass. The lignin may be e.g. Kraft lignin, organosolv lignin, and/or lignin obtainable from an alkali/soda process, a LignoBoost process, and/or a lignosulfonate process. Some types of lignin, such as lignin from a lignosulfonate process, may however be water soluble. Such lignins may not be (well) suited for the production of lignin nanoparticles.

The composition of the lignin and of the lignin nanoparticles may naturally depend on the source of the lignin. For example, softwood lignin may mainly comprise G units, hardwood lignin may comprise more S than G units, and grass lignin may comprise G, S, and H units. Further, the process used to obtain the lignin may have an effect on the structure of the lignin. The composition of the lignin may thereby also affect e.g. the efficiency of laccase enzymes used and the resulting properties of the lignin-hemicellulose hybrid nanoparticles or of the lignin nanoparticles.

The lignin-hemicellulose hybrid nanoparticles may comprise e.g. at least 0.01%, or at least 0.1% (w/w) of hemicellulose. The lignin-hemicellulose hybrid nanoparticles may comprise e.g. about 0.01-20% (w/w) of hemicellulose, or about 0.1-15% (w/w) of hemicellulose, or about 0.01-5% (w/w) of hemicellulose. The lignin-hemicellulose hybrid nanoparticles may in some embodiments comprise e.g. at least 2% (w/w) of hemicellulose. The lignin-hemicellulose hybrid nanoparticles may in some embodiments comprise e.g. at least 5%, or at least 10% (w/w), or about 2-20% (w/w) of hemicellulose.

The exact proportion or amount of the hemicellulose may however not be particularly important or limited, at least in some embodiments. For example, pyrolysis gas chromatography mass spectrometry (Py-GCMS, described in detail e.g. in the Examples) may be used to indirectly determine the proportion of the hemicellulose, but it does not, at least in some embodiments, necessarily have fully quantitative precision. Therefore, the proportion of the hemicellulose may be determined by quantifying the monosaccharide composition of the lignin-hemicellulose hybrid nanoparticles by acid methanolysis and subsequent analysis by gas chromatography.

The monosaccharide composition of the lignin-hemicellulose hybrid nanoparticles may depend on the proportion of the hemicellulose, as well as e.g. on the source and composition of the hemicellulose and on the source and composition of the lignin.

The monosaccharide composition of the lignin-hemicellulose hybrid nanoparticles may comprise at least 0.01%, or at least 0.1%, or at least 1% (w/w) of mannose, and/or at least 0.5%, or at least 1%, or at least 2% (w/w) of xylose. The monosaccharide composition of the lignin-hemicellulose hybrid nanoparticles may comprise about 0.01-5%, or about 0.1-5%, or about 1-5% (w/w) of mannose, and/or about 0.5-6%, or about 1-6%, or about 2-6% (w/w) of xylose.

The monosaccharide composition of the lignin-hemicellulose hybrid nanoparticles may comprise at least 0.01%, or at least 0.1%, or at least 1% (w/w) of mannose, and/or at least 1% (w/w) of xylose, when the lignin is softwood lignin, for example softwood Lignoboost lignin.

The monosaccharide composition of the lignin-hemicellulose hybrid nanoparticles may comprise at least 0.01%, or at least 0.1%, or at least 1% (w/w) of mannose, and/or at least 1% (w/w) of xylose, when the lignin is grass lignin.

The monosaccharide composition of the lignin-hemicellulose hybrid nanoparticles may comprise at least 0.01%, or at least 0.1%, or at least 1% (w/w) of mannose, and/or at least 2% (w/w) of xylose, when the lignin is hardwood lignin.

The hemicellulose may increase the hydrophilicity of the lignin-hemicellulose hybrid nanoparticles. The contact angle of the lignin-hemicellulose hybrid nanoparticles may be used as a measure of the hydrophobicity.

The lignin-hemicellulose hybrid nanoparticles may, at least in some embodiments, have a contact angle of e.g. 35° or smaller. For certain applications, more hydrophilic lignin-hemicellulose hybrid nanoparticles, e.g. those having a contact angle of e.g. 35° or smaller, may be beneficial.

The contact angle may be measured by using thin films from the lignin-hemicellulose nanoparticles for spin coating on silicon wafers. Three different concentrations (0.5, 1, and 1.5 mg/ml) of LNPs may be selected to double coat each silicon wafer, and the best coverage on the silicon wafer was for 1 mg/ml nanoparticles, which concentration was then used for the measurements. To prepare the spin-coated specimens for contact angle measurements, silicon wafers may be cut into 1.5 cm×1.5 cm square substrates, followed by plasma cleaning before spin-coating. An anchoring layer of Poly-L-lysine (PLL) may be applied to facilitate the physisorption of nanoparticles onto the substrate at 2000 rpm for 90 seconds, followed by nanoparticle layer deposition using the same parameters. A second deposition of nanoparticles may be applied by spin coating again under the same conditions. The contact angle may be measured using a suitable optical contact angle meter, such as KSV CAM 200 Optical Contact Angle Meter.

According to a second aspect, a method for producing and/or modifying lignin nanoparticles is provided. The method may comprise contacting a laccase enzyme with the lignin nanoparticles, thereby modifying one or more properties of the lignin nanoparticles.

According to the second aspect, lignin nanoparticles are also provided.

With the method, it is possible to utilize the crosslinking and/or polymerization ability of laccase enzymes, such as certain fungal laccase enzymes, to crosslink lignin and optionally hemicelluloses or other functional molecules, such as proteins.

One or more properties of the lignin nanoparticles may thus be modified.

The method may not require small molecular weight redox mediators or harsh and energy-demanding methods for modifying the lignin nanoparticles.

The method may allow for modifying the surface properties of the lignin nanoparticles to develop functional particles or particles with otherwise improved properties.

It may, additionally or alternatively, be possible to obtain lignin nanoparticles that are relatively homogeneous and have a desired particle size distribution.

It is also possible to use technical lignins in the method.

Not to be bound by theory, the laccase enzyme may catalyse the crosslinking and/or polymerisation of the lignin at least partially, for example to thereby increase the average or median size of the lignin nanoparticles.

The laccase enzyme may catalyse the oxidation of phenolic groups of the lignin at least partially, thereby modifying the one or more properties of the lignin nanoparticles, such as one or more surface properties of the lignin nanoparticles. The laccase enzyme may catalyse the oxidation of phenolic groups of the lignin such that at least 20%, or at least 30%, or at least 40%, or at least 50% of the phenolic groups of the lignin contained in the lignin nanoparticles are oxidized. The proportion of the phenolic groups that are oxidized may depend on the laccase enzyme used and on the type and/or source of the lignin. For example, in softwood lignin, the laccase enzyme may cause more dimerization than in hardwood lignin. With certain analytical methods such as pyrolysis, the proportion of oxidized phenolic groups may not include dimerization. The proportion of the oxidized phenolic groups may be measured e.g. by pyrolysis using an EGA/PY3030D Multishot pyrolyzer by carrying the pyrolysis out at 500° C. for 1 min with an interface temperature of 320° C. Pyrolysis products may be injected on the column via split injection (at 300° C.) with a split ratio of 25, and helium may be used as carrier gas with constant flow at 1.5 mL min⁻¹. The GC oven may be programmed from 70° C. (2 min) to 270° C. at 5° C. min⁻¹ and held at 270° C. for 15 min. MS detection may be used with EI at 70 eV, a source temperature of 250° C., a scan range of m/z 45-450 and a scan rate of 4.0 scans sec⁻¹.

However, the reactions involved in the laccase treatment may be complex.

It may be possible to tailor surface properties or other properties of the lignin nanoparticles e.g. to a particular application or end use.

The composition of the lignin in lignin nanoparticles may be considered to be different from lignin as such. Not to be bound by theory, it may be that in lignin nanoparticles, more hydrophilic parts of the lignin point towards the interface, so crosslinking of the lignin may be more difficult. For example, in lignin nanoparticles, aromatic rings may typically be directed towards the core of the lignin nanoparticle, and hydroxyl groups on or towards the surface of the lignin nanoparticle, rendering the cross-linking more difficult. Lignin nanoparticles also typically have a negative charge, which makes cross-linking more difficult.

In some embodiments, the laccase enzyme may be such that is not capable of oxidizing hemicellulose and/or proteins. In other words, the laccase enzyme may not be capable of oxidizing the carbohydrate part of the hemicellulose. It may not be desirable for the laccase enzyme to chemically alter the structure of the hemicellulose and/or proteins or other functional molecules.

Various laccase enzymes may be commercially or otherwise available.

The laccase enzyme may have an amino acid sequence comprising or consisting of a sequence that is at least 90%, or at least 95%, or 98%, or 100% identical to at least one of the sequences set forth in SEQ ID NO: 1 (DsLcc4), SEQ ID NO: 2 (PrLac2), SEQ ID NO: 3 (TpLccMut), SEQ ID NO: 4 (CcLcc9), or SEQ ID NO: 5 (OrLcc2Mut).

The laccase enzyme may have an amino acid sequence comprising or consisting of a sequence that is at least 90%, or at least 95%, or 98%, or 100% identical to the sequences set forth in SEQ ID NO: 1 (DsLcc4). This sequence is derived from Dichomitus squalens (accession number in the Mycocosm 148140 https://mycocosm.jgi.doe.gov/cgi-bin/dispGeneModel?db=Dicsql&id=148140). The sequence corresponds to the mature region of the protein (without signal peptide or N-terminal methionine).

The laccase enzyme may have an amino acid sequence comprising or consisting of a sequence that is at least 90%, or at least 95%, or 98%, or 100% identical to the sequence set forth in SEQ ID NO: 2 (PrLac2). This sequence is derived from Phlebia radiata (accession number in GenBank CAI56705.1 https://www.ncbi.nlm.nih.gov/protein/CAI56705.1). The sequence corresponds to the mature region of the protein (without signal peptide or N-terminal methionine).

The laccase enzyme may have an amino acid sequence comprising or consisting of a sequence that is at least 90%, or at least 95%, or 98%, or 100% identical to the sequence set forth in SEQ ID NO: 3 (TpLccMut). This laccase enzyme is mutated from the wild type Trametes pubescens sequence (accession number 11418 in the Mycocosm https://mycocosm.jgi.doe.gov/pages/search-forgenes.jsf?organism=Trapub1, mature region of the protein (without signal peptide or N-terminal methionine)). The sites of the mutations are F162A/A240P/Q282E/S427N/A464T. The amino acid sequence of the laccase enzyme may thus comprise or consist of a sequence that is at least 90%, or at least 95%, or 98%, or 100% identical to the sequence set forth in SEQ ID NO: 3 (TpLccMut), wherein the sequence comprises the following amino acids in the indicated positions: 162A, 240P, 282E, 427N, and/or 464T. These positions correspond to the positions of the sequence set forth in SEQ ID NO: 3 (i.e. positions 162, 240, 282, 427, 464 in SEQ ID NO: 3).

The laccase enzyme may have an amino acid sequence comprising or consisting of a sequence that is at least 90%, or at least 95%, or 98%, or 100% identical to the sequence set forth in SEQ ID NO: 4 (CcLcc9). This laccase enzyme seems to be well suited for the methods disclosed herein. This sequence is derived from Coprinopsis cinerea (https://www.ncbi.nlm.nih.gov/search/all/?term=BK004119). The sequence corresponds to the mature region of the protein (without signal peptide or N-terminal methionine).

The laccase enzyme may have an amino acid sequence comprising or consisting of a sequence that is at least 90%, or at least 95%, or 98%, or 100% identical to the sequence set forth in SEQ ID NO: 5 (OrLcc2Mut, also referred to herein as OrLcc2-D206N). This laccase enzyme is mutated from the wild type Obba rivulosa Lac2 sequence (accession number JQ027727 in the genBank). The sequence corresponds to the mature region of the protein (without signal peptide). The site of the mutation is D206N. The amino acid sequence of the laccase enzyme may thus comprise or consist of a sequence that is at least 90%, or at least 95%, or 98%, or 100% identical to the sequence set forth in SEQ ID NO: 5, wherein the sequence comprises an N (asparagine) residue in the position 206. This position correspond to the position 206 of the sequence set forth in SEQ ID NO: 3.

To determine the extent of identity of two sequences, methods of alignment are well known in the art. Thus, the determination of percent identity between any two sequences can be accomplished using a mathematical algorithm such as the algorithm described by Lipman and Pearson (Science 1985, 227(4693), 1435-1441). For example, the ClustalW or ClustalΩ software may be used for the alignment. The sequences set forth in this specification are provided as non-limiting examples. The percentage identity may be relative to the full length of the reference sequence to which the sequence in question is compared, or based on a partial alignment.

Various methods for forming the lignin nanoparticles, which already as such may be considered to be a form of lignin nanoparticles, are available. The morphology and/or polydispersity of the lignin nanoparticles and/or of the lignin-hemicellulose hybrid nanoparticles may vary depending on the source of the lignin and the method for forming the lignin nanoparticles.

The method may comprise forming the lignin nanoparticles by dissolving lignin in a mixture comprising acetone and water and contacting the mixture comprising the dissolved lignin with water, thereby obtaining the lignin nanoparticles. This manner of forming the lignin nanoparticles may provide lignin nanoparticles that are mainly spherical or essentially spherical. The lignin nanoparticles obtained also appear to have a lignin structure that is not significantly altered. This manner of forming the lignin nanoparticles may also provide lignin nanoparticles that are relatively small and homogeneous. The mixture may comprise acetone and water in a ratio of 3:1, or in a ratio in the range of 4:1 to 2:1. The water may be considered to be an anti-solvent that precipitates the dissolved lignin into lignin particles, which may be utilized as the lignin nanoparticles.

The method may comprise forming the lignin nanoparticles by acid precipitation. Such as method may comprise forming the lignin nanoparticles by dissolving lignin in an alkaline solution, such as an NaOH solution, and contacting the alkaline solution comprising the dissolved lignin with an acid or a solution thereof, such as HCl, thereby obtaining the lignin nanoparticles.

The method may comprise forming the lignin nanoparticles by dissolving lignin in a solution comprising 70% ethanol and contacting the solution comprising the dissolved lignin with water, thereby obtaining the lignin nanoparticles.

The method may further comprise grafting a functional molecule to the lignin nanoparticles. After the laccase reaction, radicals formed on the lignin structure may be capable of reacting with a variety of molecules, for example proteins, lipids, phenolic compounds (for example, tannic acid), and/or carbohydrates.

The functional molecule may be at least one of a protein, a lipid, a phenolic compound, or a carbohydrate. The functional molecule may comprise a group capable of reacting with the at least partially oxidized lignin of the lignin nanoparticles. For example, the protein may comprise an exposed tyrosine residue capable of reacting with the at least partially oxidized lignin of the lignin nanoparticles. The functional molecule may naturally be selected based on its properties and/or properties desired for the grafted lignin nanoparticles.

The functional molecule may be grafted to the lignin nanoparticles by contacting the functional molecule with the lignin nanoparticles. This may be done after the laccase enzyme has been contacted with the lignin nanoparticle, and/or simultaneously. Suitable conditions may be selected e.g. such that the reaction between the functional molecule and the lignin may take place.

The lignin nanoparticles may thus further comprise a functional molecule grafted to the lignin nanoparticles and/or a coating covering the lignin nanoparticles.

The method may further comprise adding a coating to surfaces of the lignin nanoparticles, covering the lignin nanoparticles with the thereby coating. The coating may comprise or be e.g. hemicellulose, but various other coatings may also be contemplated.

The method may further comprise adding hemicellulose to the lignin nanoparticles, thereby covering the lignin nanoparticles with hemicellulose and optionally crosslinking the hemicellulose to the lignin nanoparticles. Lignin nanoparticles thereby obtainable may be considered to be lignin-hemicellulose hybrid nanoparticles.

The lignin nanoparticles may be stable in water or an aqueous solution for at least 4 weeks. The pH of the aqueous solution may be about 7. The lignin nanoparticles may be stable in the aqueous solution for at least 4 weeks in storage, for example at a temperature of about 4° C. The lignin nanoparticles may be stable in the aqueous solution longer, for at least 7 weeks, or at least one month, or at least 2 months, or at least 3 months, or at least 6 months.

The lignin nanoparticles may be obtainable or obtained by the method according to one or more embodiments described in this specification.

The ζ-potential of the lignin nanoparticles in water may be e.g. −45 mV or lower, or e.g. in the range of −60-−45 mV. The ζ-potential may be measured as set out above in this specification.

In the context of this specification, the term “lignin nanoparticle” should not be understood as necessarily being limited to any particular particle size. It may be understood as referring to lignin particles having e.g. a particle size, for example a median or average particle size, in the range of 1-1000 nm.

The average or median particle size of the lignin nanoparticles may be in the range of 100-500 nm. The average or median particle size may be measured as set out above in this specification. The average or median particle size may be the average or median particle size of the lignin nanoparticles in water or an aqueous solution having a pH of about 6-7. Laccase-treated lignin nanoparticles may have a relatively high average or median particle size, because they may tend to crosslink with each other through the formation of a C—C type of linkage.

The polydispersity index (PDI) of the lignin nanoparticles may be 0.2 or lower, or 0.15 or lower, or in the range of 0.01-0.2. However, with certain types of lignin, in particular when treated with a laccase enzyme, the PDI may be higher. The polydispersity index (PDI) of the lignin nanoparticles may, in some embodiments, be 0.60 or lower, or in the range of 0.01-0.60.

In some embodiments, the lignin nanoparticles may be considered to be stable in the water or the aqueous solution, when their polydispersity index remains at e.g. 0.2 or lower, or 0.15 or lower, in the water or the aqueous solution for the at least 4 weeks (or other time period as set out in this specification).

In some embodiments, the lignin nanoparticles may be considered to be stable in the water or the aqueous solution, when the average or median particle size of the lignin nanoparticles remains in the range of 100-500 nm, or in the range of 100-200 nm in the aqueous solution for the at least 4 weeks (or other time period as set out in this specification).

The lignin in the lignin nanoparticles may be any lignin described in this specification.

A composition or product comprising the lignin-hemicellulose hybrid nanoparticles according to one or more embodiments described in this specification and/or the lignin nanoparticles one or more embodiments described in this specification is also provided. The composition or product may be e.g. a pharmaceutical composition; a wound dressing; a hydrogel; a microneedle; a stabilizer (e.g. an emulsion stabilizer); a film; a coating; a sunscreen protector; a lotion; a plastic replacement, such as a replacement for microplastic particles for cosmetic and personal hygiene products; or a food packaging product, such as a food packaging film.

The use of the lignin-hemicellulose hybrid nanoparticles according to one or more embodiments described in this specification or the lignin nanoparticles one or more embodiments described in this specification in a pharmaceutical composition; a wound dressing; a hydrogel; a microneedle; a stabilizer (e.g. an emulsion stabilizer); a film; a coating; a sunscreen protector; a lotion; or a food packaging product, such as a food packaging film, is also disclosed.

A laccase enzyme is also provided, wherein the laccase enzyme has an amino acid sequence comprising a sequence that is at least 90%, or at least 95%, or at least 98%, or 100% identical to a sequence set forth in SEQ ID NO: 3 (TpLccMut). The amino acid sequence of the laccase enzyme may comprise or consist of a sequence that is at least 90%, or at least 95%, or 98%, or 100% identical to the sequence set forth in SEQ ID NO: 3 (TpLccMut), wherein the sequence comprises the following amino acids in the indicated positions: 162A, 240P, 282E, 427N, and/or 464T. These positions correspond to the positions of the sequence set forth in SEQ ID NO: 3 (i.e. positions 162, 240, 282, 427, 464 in SEQ ID NO: 3).

### EXAMPLES

Reference will now be made in detail to various embodiments, an example of which is illustrated in the accompanying drawings.

The description below discloses some embodiments in such a detail that a person skilled in the art is able to utilize the embodiments based on the disclosure. Not all steps or features of the embodiments are discussed in detail, as many of the steps or features will be obvious for the person skilled in the art based on this specification.

### Example 1

### Materials

Three lignin samples were selected for this study. Softwood kraft Lignoboost was provided by Stora Enso (Finland). Hardwood birch lignin (Betula L.) was isolated using the BLN process, and obtained from CH Bioforce Oy (Finland). Protobind 1000 was extracted from wheat straw by the soda process and acquired from GreenValue SA (Switzerland). Spray dried (sd) spruce Galactoglucomannan (GGM) and birch Glucuronoxylans (GX) were obtained from Luke (Finland). Both hemicelluloses were submitted to an ethanol precipitation (ep) approach to reduce their lignin content. Acetone for HPLC (≥99.9%), citric acid monohydrate were acquired from Sigma-Aldrich (Finland). Myceliophthora thermophila (MtL, Novozym® 51003) was purchased from Novozymes A/S (Denmark).

Heterologous expression of fungal laccases: Basidiomycete laccases, namely Coprinopsis cinereus (CcLcc9; GenBank accession no. BK004119), Obba rivulosa (OrLcc2-D206N named here OrLcc2Mut), Trametes pubescens (TpLccMut) and Dichomitus squalens (DsLcc4; GenBank accession no. TBU29213) were purchased in pPICZαA expression vector (GenScript, NJ, USA). Laccase variant TpLccMut was designed by site-directed mutagenesis based on the cDNAs encoding TpLcc2 (GenBank accession no. OJT12045). The sequence was mutated at the following sites: F162A/A240P/Q282E/S427N/A464T. The Phlebia radiata (PrLcc2; GenBank accession no. CAI56705) CDNA was cloned into pPICZαA expression vector (Invitrogen). Heterologous expression of fungal laccases was performed as described previously (Hildén et al., Appl. Microbiol. Biotechnol. 2013, 97, 1589). The linearized plasmid constructs were transformed into Pichia pastoris X-33 competent cells by electroporation and the transformants were selected on yeast extract-peptone [YEP; 1% (wt/vol) yeast extract (Labema, Finland), 2% (wt/vol) peptone (Labema, Finland)], 2% (wt/vol) glucose and sorbitol (182.2 g L⁻¹) containing agar plates supplemented with zeocin (100 μg mL⁻¹). The best laccase-producing transformants were chosen by using 2,2′-azino-bis(3-ethylbenzathiazoline-6-sulfonate) (ABTS)-plate assay. The selected transformants were cultivated in YEP liquid medium supplemented with 1% (w/v) glycerol at 28° C. with shaking (200 rpm) until OD₆₀₀ was approximately 6-8. The cells were pelleted by centrifugation at 4° C., 1500 g for 5 min and obtained pellets were washed with phosphate-buffered saline (PBS; 137 mM NaCl, 2.7 mM KCl, 10 mM Na₂PO₄, 1.8 mM KH₂PO₄). The cell pellets were resuspended in buffered minimal medium (BMM; 100 mM potassium phosphate, pH 6, 1.34% yeast nitrogen base (YNB), 4*10-5% biotin) supplemented with 0.3 mM CuSO₄ and 0.5% Tween-20. Laccase expression was controlled daily by 0.5% (v/v) methanol addition and the induction was continued for 5 days. The extracellular laccase activity was followed daily by 2,6-dimethoxyphenol (2,6-DMP) as substrate. The cultivation supernatant was collected by centrifugation (3500 g, 15 min, 4° C.) and phenylmethylsulfonyl fluoride was added to final concentration of 0.1 M to inhibit protease activity. The cultivation medium was concentrated by using Amicon® pressurized ultrafiltration unit (Millipore; Stirred Cell 400 mL) and with Spin-X® UF 20 mL concentrator (Corning) with 10 kDa cut-off till the final volume of 3-5 mL.

### Preparation of Lignin Nanoparticles

The acetone nanoprecipitation approach used to prepare LNPs was previously reported by Figueiredo et al., ChemSusChem 2021, 14, 4718). Briefly, 2 g of technical lignins were dissolved in 200 ml of acetone/water 3:1 (v/v) mixtures and stirred for 3 h, followed by their filtration using a glass microfiber filter (Whatman GF/F, pore size 0.7 μm). The obtained solution was rapidly poured into 400 mL of MilliQ-water under vigorous stirring. Acetone was further removed by evaporation under reduced pressure at 40° C. to obtain the LNPs dispersions. Finally, the LNP suspensions were centrifuged for 15 min at 50000 g, and redispersed with MilliQ-water using ultrasonication (Branson digital sonicator) at a frequency of 20 kHz, 30% oscillation amplitude (100 W) for 60 sec.

Laccase-Induced Oxidation of LNPs and Cross-linking with Hemicelluloses

All hemicelluloses (sdGGM, epGGM, sdGX, and epGX) were previously dissolved in 25 mM citric acid pH 5 at concentration of 1, 2, 5, and 10 mg/mL, overnight, and further centrifuged for 10 min at 20000 g to remove the undissolved hemicellulose. The crosslinking reactions were performed in aqueous dispersions of LB-, PB-, and BB-LNPs in 25 mM citric acid pH 5 at final concentration of 1 mg/mL, containing different concentrations of hemicelluloses, which were further diluted in the reaction to 0.5, 1, 2.5, and 5 mg/mL. Finally, all the laccases (MtL, DsLcc4, PrLac2, TpLccMut CcLcc9, OrLcc2Mut) at dosage of 1000 nKat per gram (nKat/g) of lignin were added to the previous mixture, and allowed to react for 24 h. Control samples were prepared in the same way, without any hemicellulose and/or laccase treatment.

After the reaction, the samples were centrifuged at 12500 g for 15 min, washed twice with MilliQ-water, and further redispersed with MilliQ-water for further analysis.

### Dynamic Light Scattering

The average hydrodynamic diameter, polydispersity index, and ζ-potential of LNPs was measured by dynamic light scattering (DLS), using a Malvern Zetasizer Nano ZS instrument (Malvern Instruments Ltd, UK). For that, the samples were diluted in MilliQ-water at a concentration of 0.5 mg/mL. Different time points (Day 1 and Week 7) were considered in order to evaluate the long term-stability of the prepared LNPs.

### Pyrolysis Gas Chromatography Mass Spectrometry (Py-GCMS)

Pyrolysis was performed with an EGA/PY3030D Multishot pyrolyzer (Frontier Laboratories, New Ulm, MN, USA) equipped with an AS-1020E Autoshot autosampler. The pyrolyzer was coupled to GC-MS using a Trace GC equipped with a DB-1701 fused-silica capillary column (30 m×0.25 mm i.d. 0.25 μm film thickness) coupled to a DSQ-II mass spectrometer (Thermo Scientific, Waltham, MA, USA). Samples were weighed using a XP6 excellence-plus microbalance (Mettler Toledo, Columbus, OH, USA). Pyrolysis of LB-, PB-, and BB-LNPs (100-120 μg) was carried out at 500° C. for 1 min with an interface temperature of 320° C. Pyrolysis products were injected on the column via split injection (at 300° C.) with a split ratio of 25, and helium was used as carrier gas with constant flow at 1.5 mL min⁻¹. The GC oven was programmed from 70° C. (2 min) to 270° C. at 5° C. min⁻¹ and held at 270° C. for 15 min. MS detection was used with EI at 70 eV, a source temperature of 250° C., a scan range of m/z 45-450 and a scan rate of 4.0 scans sec⁻¹. Compounds were identified by comparing retention time and mass spectrum with standards, the NIST library.

### Stability of LNPs-Hemicellulose Complexes at pH 3

After reacting the LNPs with hemicellulose, the stability of the LNPs-hemicellulose complexes was evaluated by incubating the LNPs at concentration of 0.5 mg/mL in 25 mM citric acid pH 3. Afterwards, the average hydrodynamic diameter and ζ-potential of the LNP suspensions was measured by DLS, using the Malvern Zetasizer Nano ZS instrument.

### LNP Dispersion Stability

The colloidal stability of LNP suspensions during storage was monitored using Turbiscan Lab Expert (Formulaction, Toulouse, France) at the wavelength of 800 nm (near-infrared light). The transmitted light intensity were measured using Turbisoft version 1.2 (Formulaction, Toulouse, France) software. Results were presented in terms of percentage of ΔTransmission, calculated by the difference of the transmitted light at predefined time point and the initial transmitted light through the LNP suspensions. The measurements were performed just after LNP preparation and up to 7 weeks storage.

### Characterization of LNPs

The anti-solvent precipitation approach may be used to accomplish spherical LNPs, with uniform size, smooth surfaces, and high colloidal stability, which enable the application of these LNPs for different fields, such as drug delivery and emulsion stabilizers.

However, the homogeneity and morphology of LNPs produced with this methodology can significantly vary according to the lignin grade and source, as they present different mass, molar phenolic hydroxyl groups, and solubility properties. An approach using acetone/water (3:1) mixture as the lignin solvent, and water as the anti-solvent, has been used to obtain the LNPs for further laccase treatment, and they were characterized by dynamic light scattering (DLS) for their hydrodynamic diameter, PDI, and ζ-potential, and the amount of phenolic units were quantified by pyrolysis gas chromatography mass spectrometry (Py-GCMS) (Table 1).

Table 1. Characterization of LB-, PB- and BB-LNPs in terms of their average size, PDI and ζ-potential using DLS (n≥3). Normalized area of the main lignin-derived monolignols (H; p-hydroxyphenyl; G, guaiacyl; S, syringyl) identified using Py-GCMS, and S/G ratio of LNPs.

| | LB-LNPs | PB-LNPs | BB-LNPs |
|---|---|---|---|
| Size (nm) | 112.5 ± 6.9 | 166.4 ± 4.7 | 177.2 ± 8.1 |
| PDI | 0.141 ± 0.015 | 0.116 ± 0.011 | 0.105 ± 0.021 |
| ζ- | −43.3 ± 1.4 | −47.2 ± 0.9 | −47.7 ± 1.2 |
| potential | | | |
| (mV) | | | |
| Total H | 7.62 | 10.37 | 2.31 |
| (%) | | | |
| Total G | 90.64 | 42.07 | 27.09 |
| (%) | | | |
| Total S | 1.74 | 47.56 | 70.60 |
| (%) | | | |
| S/G ratio | 0.02 | 1.13 | 2.61 |

The hydrodynamic diameter of LB-LNPS was found to be slightly smaller than that of the PB- and BB-LNPs, which could be due to non-covalent forces acting during the self-assembling process of the three technical lignins obtained from different sources that exhibit different molar mass. The increased hydrophobic interactions in LB lignin presenting higher molar mass drives the formation of smaller LNPs than PB- and BB-LNPs. In addition, the π-π interactions between guaiacyl units on the LB lignin are stronger than the interactions between the syringyl units on PB and BB lignins, leading to the production of more packed LB-LNPs with smaller size than the PB- and BB-LNPs. The as-prepared LNPs exhibited PDI values lower than 0.15, suggesting that the LNPs are homogeneous and monodispersed. Given by the ζ-potential values, the surface charge of LNPs was negative due to the presence of carboxylic groups on the LNP surface, leading to the stabilization of the LNPs in colloidal dispersion caused by the electric double-layer repulsion.

Py-GCMS was performed in order to quantify the percentage of monolignols and S/G ratio in the three LNPs obtained from different technical lignins. The proportion of the lignin-derived monolignols and linkages between them can vary with the lignin source, being the softwood lignin composed mostly by G units, the hardwood lignin comprises more S than G units, while the grass lignin presents the G, S, and H units. As expected, the LB-LNPs obtained from softwood presented the highest amount of G units (ca. 90%), whereas the PB- and BB-LNPs exhibited about 42 and 27%, respectively. Conversely, the hardwood birch-derived BB-LNPs and the Sarkanda grass-derived PB-LNPs displayed 70 and 47% of S units, respectively, while the LB-LNPs had almost no S units. Therefore, the S/G ratio in hardwood BB-LNPs (was substantially higher than in the softwood LB-LNPs (0.02). Moreover, the Sarkanda grass-derived PB-LNPs presented an S/G ratio of 1.13, as it comprises similar percentage of G and S units in addition to the 10% of H units. The S/G ratio is considered an important parameter in the biorefinery process because it reflects on the acidic or enzymatic hydrolysis of biomass to produce fermentable sugars. In addition, S/G ratio might also have an impact on the laccase efficiency on the crosslinking and polymerization of LNPs.

### Size of Hemicellulose-Coated LNPs

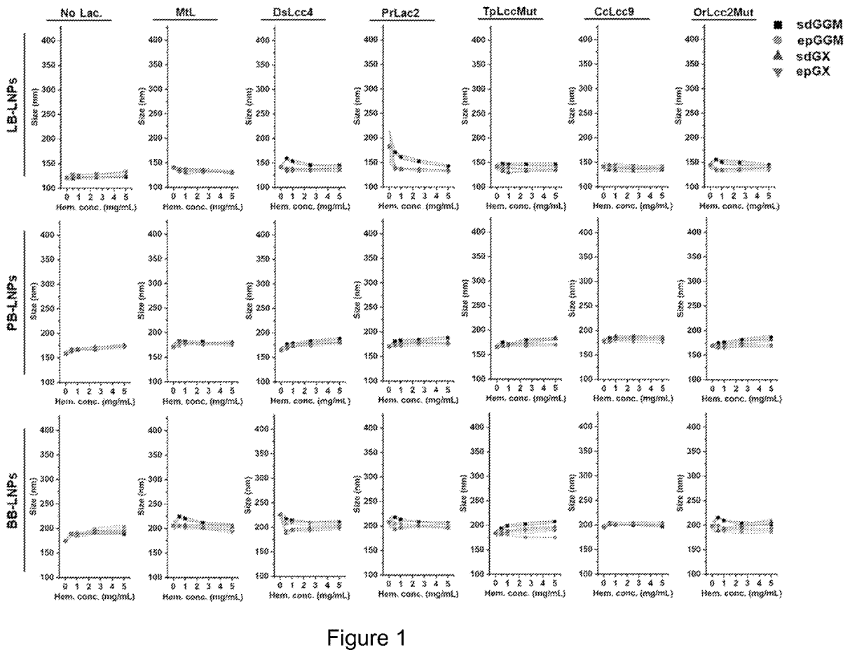

The size of LNPs was measured in order to evaluate the effect of the hemicellulose coating with and without the laccase treatment (FIG. 1). For the softwood-derived LB-LNPs, the size of the LNPs that were not treated with laccases remained similar as the hemicellulose concentration in the reaction increase.

The laccase-treated LB-LNPs experienced an increase in particle size when compared with non-treated LNPs, as consequence of the intercross-linking of particles/lignin due to the formation of oxidized dimeric products derived from the polymerization of G-type rich softwood LNPs. However, after adding the hemicelluloses in the reaction, the size of LNPs decreased with the increase on the hemicellulose concentration, which might indicate that the hemicellulose coating can stabilize the LNP surface. As for the PB- and BB-LNPs, their size after laccase treatment did not increase substantially, due to the higher content on S-type units that have the 5-position on the aromatic ring occupied by the methoxy group, preventing the LNP polymerization. In addition, the size of the PB- and BB-LNPs experienced a slight increase after adding the hemicelluloses at different concentrations. The LNP size remained similar after 7 weeks of storage at 4° C.

### Surface Charge of Hemicellulose-Treated LNPs

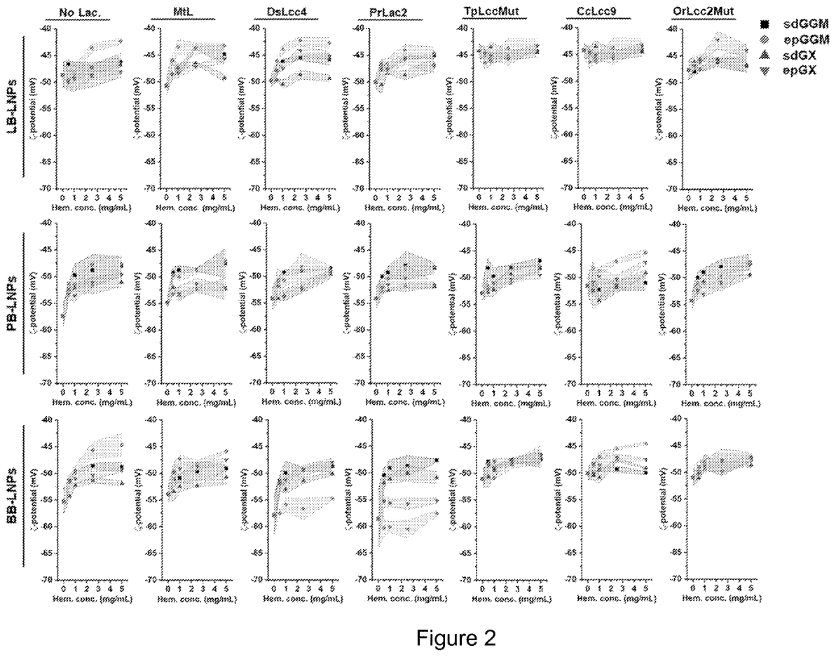

The surface charge of LNPs, here given by their ζ-potential values, was quantified in order to evaluate the effect of the concentration and type of hemicellulose on the LNP coating after reaction (FIG. 2). Generally, the ζ-potential values increased as the hemicellulose concentration in the reaction increased, which indicates that the hemicellulose coating was successful. In addition, the trends on the zeta values of LNP remained similar after 7 weeks of storage at 4° C.

### Stability of LNPs-Hemicellulose Complexes at pH 3

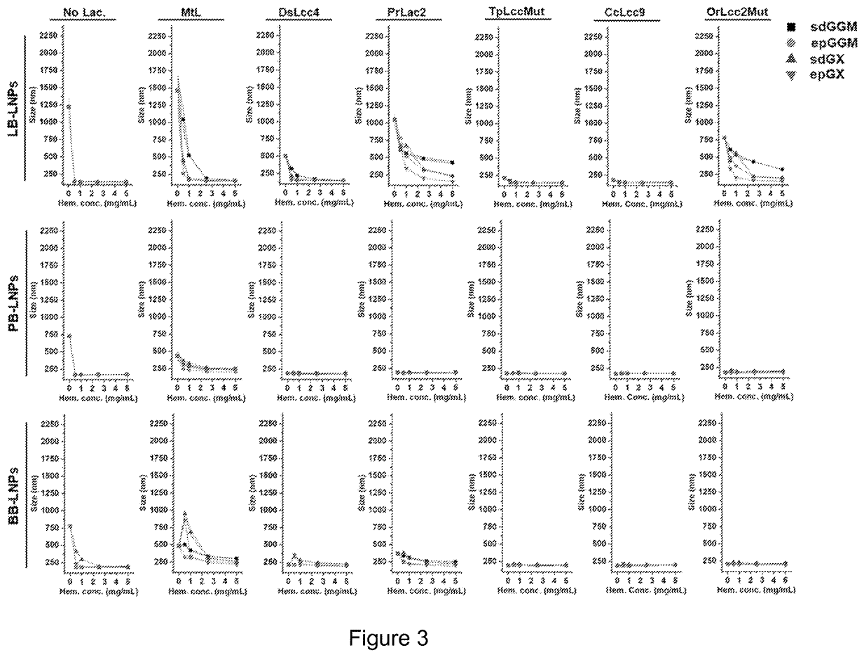

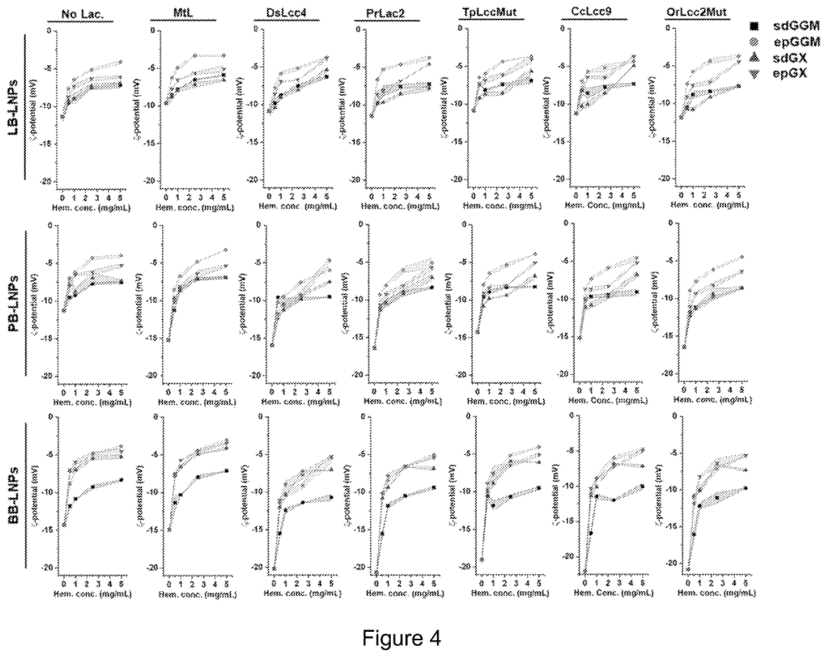

The stability of LNPs at wide range of pH is desired to increase the potential application of LNPs in different areas, including biomedical and food sciences. Here, the stability of the prepared LNPs was assessed after their incubation with 25 mM citric acid pH 3, in terms of average size (FIG. 3) and ζ-potential (FIG. 4).

The size of bare LNPs without any treatment tend to radically increase at acidic pH to over 700 nm (FIG. 3), due to their aggregation when the pH gets closer to the isoelectric point and the carboxyl groups become protonated, inducing the intermolecular hydrogen bonding between particles. Generally, the treatment of LNPs with the fungal laccases resulted in an improved stability of the LNP surface, leading to a decrease in the LNP size compared to untreated control, in particular for the PB- and BB-LNPs. However, the LNP size dramatically decreased after adding the hemicelluloses on the reaction, according to the concentration of hemicellulose added. Interestingly, hemicelluloses seemed to be adsorbed on non-laccase treated LNP surface, probably due to non-covalent interactions between functional groups on both lignin and hemicelluloses, even at the lowest concentration of hemicelluloses.

Regarding the ζ-potential values (FIG. 4), and without any treatment, the surface charge of the LNPs tend to increase as the pH gets highly acidic, as a consequence of the protonation of the carboxylic groups, and consequently, the LNPs get aggregated. Generally, the ζ-potential values increased even further as a result of the presence of the hemicelluloses on the LNP surface. However, even when the surface charge gets close to neutral, the LNPs do not experience aggregation due to the presence of hemicelluloses on the LNP surface.

### Colloidal Stability of LNPs-Hemicellulose Complexes

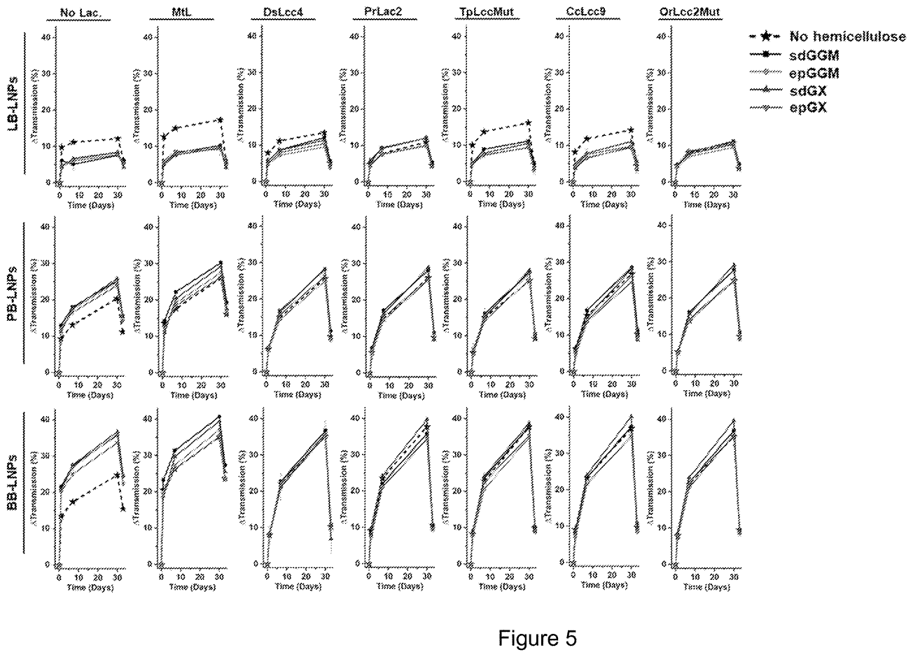

The colloidal stability of LNPs in MQ-water was also evaluated using Turbiscan, by measuring the variation of the light transmitted through the LNP suspensions over time (FIG. 5). Generally, the light transmitted through the LNP suspensions varied according to the size of the LNPs, i.e. the percentage of transmission was higher for the both PB- and BB-LNPs than for LB-LNPs. In addition, the light transmission was improved after hemicellulose coating of the laccase treated LNPs, in particular for LB-LNPs, as the non-coated LNPs presented slightly higher particle size after laccase treatment, compared to the control LB-LNPs without laccase incubation. After storage, the particles can be easily redispersed after gentle mixing, showing light transmission values similar to day of storage.

### Example 2

LNPs were prepared by anti-solvent precipitation using three different technical lignins as starting material: hardwood birch lignin (BLN process), wheat straw/Sarkanda grass Protobind™ 1000 (alkali), and softwood LignoBoost (kraft). The main aim was to systematically characterize the laccase-assisted oxidation of LNPs with different percentages of monolignols and S/G ratios, using five fungal laccases produced in house, and compare their effect with two commercial laccases, without using any mediators. The oxidation mechanism was evaluated in terms of oxidized-derived pyrolysis compounds, changes in the absorbance spectrum, and phenolic content after treatment. The size and surface charge of LNPs was determined to evaluate the effect of laccase treatment on the physicochemical characteristics of LNPs.

Materials: Three lignin samples were selected for this study. Softwood kraft Lignoboost was provided by Stora Enso (Finland). Hardwood birch lignin (Betula L.) was isolated using the BLN process, and obtained from CH Bioforce Oy (Finland). Protobind 1000 was extracted from wheat straw by the soda process and acquired from GreenValue SA (Switzerland). Acetone for HPLC (≥99.9%), citric acid monohydrate were acquired from Sigma-Aldrich (Finland), and Trametes versicolor (TvL) from Sigma-Aldrich (Germany). Myceliophthora thermophila (MtL, Novozym® 51003) was purchased from Novozymes A/S (Denmark).

The heterologous expression of fungal laccases (DsLcc4, PrLac2, TpLccMut, CCLcc9, OrLcc2Mut) was performed as described in Example 1.

Preparation of lignin nanoparticles: The LNPs were prepared using the acetone nanoprecipitation approach as described in Example 1.

Laccase treatment of lignin nanoparticles: The laccase reactions were performed in aqueous dispersions of LB-, PB-, and BB-LNPs in a final volume of 1.5 mL. For these reactions, all the laccases were incubated with the 1 mg mL⁻¹ of LNPs dispersed in 25 mM citric acid pH 5, at laccase dosage of 100, 500, and 1000 nKat per gram (nKatg⁻¹) of lignin. Laccase activities were calculated using 2,6-Dimethoxyphenol as substrate. The reaction was carried out at room temperature with ambient air (O₂) circulation under gentle stirring, for 1 and 24 h. Control samples were also prepared in the same way, without any laccase treatment.

After the reaction time, the samples were washed twice with MilliQ-water and centrifuged at 12500 g for 15 min, and the oxidized-LNPs were redispersed with MilliQ-water for further analysis.

Dynamic light scattering: The average hydrodynamic diameter, polydispersity index, and ζ-potential of LNPs was measured by dynamic light scattering (DLS), using a Malvern Zetasizer Nano ZS instrument (Malvern Instruments Ltd, UK). For that, the samples were diluted in MilliQ-water at a concentration of 500 μg mL⁻¹.

Pyrolysis gas chromatography mass spectrometry (Py-GCMS): Py-GCMS was performed as described in Example 1.

Quantification of the absorbance of LNPs: The oxidized dimeric products derived from the lignin can have an increased absorbance at 515 nm. In that way, 200 μL of LNP suspensions at concentration of 100 μg mL⁻¹ were placed in 96-well plate and the UV-Vis spectra of the lignin was measured between 400 and 800 nm using a Varioskan Flash plate reader (Thermo Fisher Scientific Inc., USA). The absorbance values at 515 nm was recorded and compared between LNP samples.

Phenolic content evaluation: The total amount of phenolic hydroxyl groups was quantified with a spectrophotometric method based on the Folin-Ciocalteu reagent. For that, 50 μL of aqueous dispersions of LNP or alkali-solubilized technical lignins (0.5 mg/ml) were diluted with 1.8 mL of MilliQ-water, and further mixed with 150 μL Folin-Ciocalteu reagent. After ca. 6 min, 500 μL of sodium carbonate solution (20%, w/w) was added, and the resulting mixtures were mixed and kept at 40° C. for 30 min. Finally, the absorbance at 760 nm of the blue-colored samples was measured using a UV-Visible spectrophotometer (UV-1800 Shimadzu) with the UV probe 2.70 software. The amount of free phenolic groups was quantified from standard curve based on vanillin (4-hydroxy-3-methoxybenzaldehyde).

### Laccase-Induced Oxidation of LNPs

Enzymes, such as laccases, have emerged as eco-friendly tools for mediating coupling reactions as an alternative to metal catalysis. Laccases can act on phenolic and polymeric aromatic compounds, inducing a variety of modifications on the lignin such as polymerization, depolymerization, demethylation, and Cα—OH oxidation. The reactive free radicals of their substrates created after laccase treatment can form dimers, oligomers via different covalent linkages, such as C—C, C—O, and C—N bonds, and the aromatic compounds can undergo ring cleavage. Several reaction parameters have shown to influence the degree of lignin polymerization during laccase treatment, including the activity and enzyme dosage, the pH of the reaction, the initial lignin concentration, and temperature.

The acidity conditions of the reaction is a crucial factor that affects both the laccase activity and the solubility of lignin. Most of the fungal laccases exhibit their maximum activity at a mild acidic pH ranging from 4 to 6. In addition, lignin is prone to dissolve at very basic pH due to the ionization of phenolic groups. Therefore, an acidic buffer (25 mM citric acid pH 5) was selected, in which the fungal laccases used presented the highest activity and the LNPs remained in a stable colloidal suspension during the reaction. The LNP content in the reaction mixture was set to 1 mg mL⁻¹ after previous optimization. Laccase dosages of 100, 500 and 1000 nKat g⁻¹ of lignin were tested to evaluate the best dosage for lignin polymerization without promoting the lignin dissolution/depolymerization, and the reaction occurred for 1 and 24 h, at room temperature. In this study, the effect of five fungal laccases produced in house (DsLcc4 (SEQ ID NO: 1), PrLac2 (SEQ ID NO: 2), TpLccMut (SEQ ID NO: 3), CcLcc9 (SEQ ID NO: 4), and OrLcc2Mut (SEQ ID NO: 5)) was studied and compared with two commercial laccases (TvL and MtL), without using a mediator. After the reaction, the laccase-treated LNPs changed color from brown to red-brown (LB-LNPs) or dark brown (PB- and BB-LNPs) due to formation of quinones. Furthermore, laccase-treated LNPs exhibited different intensity according to the laccase dosage and incubation time, i.e. the higher the laccase dosage and the residence time, the more reddish or darker the LNP suspensions became.

### Pyrolysis-GCMS of Laccase-Treated LNPs

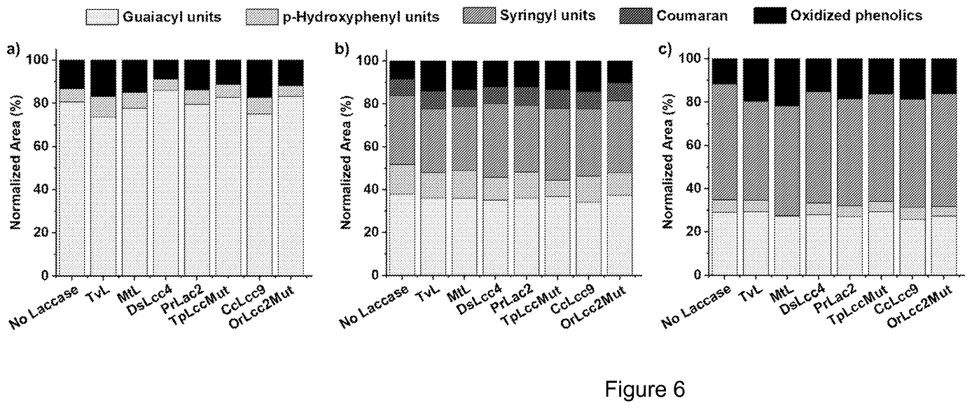

Previous studies carried out on lignin model compounds and laccases have revealed that dimeric β-1 and β-O-4 phenolic compounds treated with laccases led to the cleavage of aryl-Cα bonds or Cα-Cβ bonds, and oxidation of Cα-OH to Cα=O. In addition, free radical polymerization reactions with monomeric guaiacyl-type model compounds can occur, with the formation of a variety of C—O bonds (β-O-4′, 4-O-5) and C—C linkages (β-β, β-5, β-1, and 5-5), along with Cα-oxidation. The modifications on the LNPs after the enzymatic treatment were evaluated by Py-GCMS, which allows the analysis of lignin by chromatographic separation and mass-spectrometric identification of the compounds released after the pyrolytic breakdown of laccase-treated LNP samples. The peak areas were normalized for the total percentage of detected products, which were classified according to the lignin unit type (i.e., G, H, S), and the presence of coumaran and oxidized-derived compounds (FIG. 6).

When analyzing the lignin-derived compounds identified in the Py-GCMS of LB-LNPs and laccase-treated LB-LNPs, the area percentage of oxidized-derived compounds slightly increased by ca. 5, 14, 28 and 31% for PrLac2, MtL, TvL, and CcLcc9, respectively, while it decreased for the other laccase types (FIG. 6a). Furthermore, the prevalence of vanillin, acetovanillone, and propiovanillone oxidized G-type compounds dramatically increased after reaction with laccase. In addition to that, the percentage of lignostilbene structures dramatically decreased after laccase treatment of LB-LNPs, which can indicate some rearrangement of the aryl-Cα interunit linkages (β-β′ and β-5′) in the lignin structure. Regarding the lignin-derived compounds of PB-LNPs and laccase-treated PB-LNPs (FIG. 6b), the area percentage of oxidized-derived compounds dramatically increased for all the laccases: TvL (65%), MtL (58%), DsLcc4 (42%), PrLac2 (40%), TpLccMut (58%), CcLcc9 (68%), and OrLcc2Mut (18%). From all the compounds identified, the percentage of both vanillin and acetovanillone oxidized G-type compounds, and syringaldehyde, acetosyringone and syringylacetone oxidized S-type compounds increased after laccase treatment. This can be ascribed to the fact that PB-LNPs presented an S/G ratio of 1.13, as the proportion of both G and S units are similarly present in the PB lignin structure, and can undergo Cα-oxidation. Lastly, the lignin-derived compounds of BB-LNPs and laccase-treated BB-LNPs (FIG. 6c), the area percentage of oxidized-derived compounds increased even more than for laccase-treated PB-LNPs: TvL (69%), MtL (89%), DsLcc4 (33%), PrLac2 (61%), TpLccMut (41%), CcLcc9 (62%), and OrLcc2Mut (40%). As previously observed, the BB-LNPs presented the highest S/G ratio of the three types of LNPs, with ca. 71% of S-type units in its structure. Consequently, the frequency of oxidized S-type compounds greatly augmented after laccase treatment, such as the syringaldehyde, homosyringaldehyde, acetosyringone, syringylacetone, and propiosyringone oxidized S-units.

Overall, these observations suggest the higher formation of Cα-oxidized S-type units during the laccase treatment of BB-LNPs, compared to the LB-LNPs rich in G-type units.

### Measurement of the Absorbance at 515 nm of LNPs

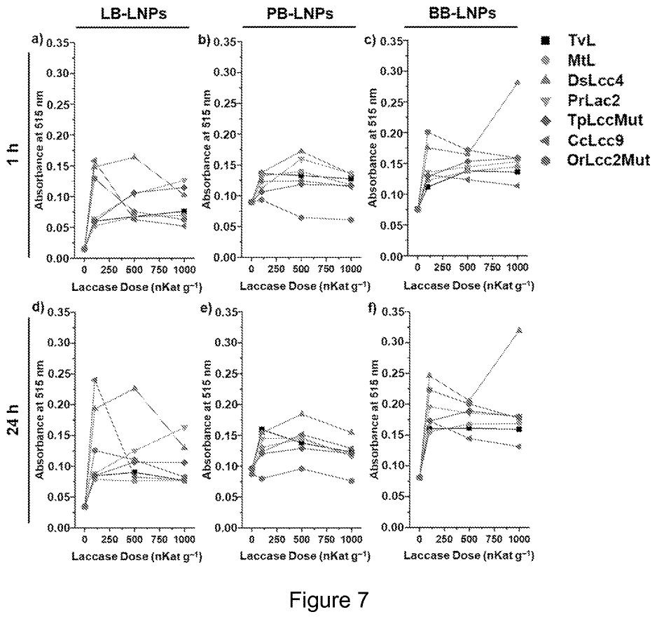

A high-throughput screening assay was developed to detect changes in the UV-visible (UV-vis) spectra of small phenolic compounds, which are resembled in the lignin structure, after laccase oxidation. When these compounds are oxidized by laccases, they present a stronger absorbance peak at around 515 nm, resulting from the appearance of oxidized dimeric products. The phenoxy radicals generated on the lignin structure after laccase treatment can spontaneously undergo a C—C coupling reaction with each other to form dimeric products. For this reason, the absorbance at 515 nm of the LNP suspensions was measured at concentration of 100 μg mL⁻¹ from the three different sources, treated with the commercial and in house produced laccases, at dosages ranging from 100 to 1000 nKat g⁻¹ at pH 5 for 1 and 24 h (FIG. 7).

Generally, all the laccase treatments induced an increase in the absorbance at 515 nm after reaction with the three LNPs, and the absolute values for the absorbance were higher after 24 h than after 1 h of reaction, suggesting a successful reaction. This augmented absorbance can be due to the presence of oxidized dimeric lignin derived products, which are derived from the phenoxy radicals generated on the lignin structure that suffer C—C coupling reaction after laccase treatment. However, as observed in FIGS. 7a, d, the softwood LB-LNPs experienced a higher absorbance than the other two LNPs rich in S-type units (FIG. 7b, c, e, f), with ca. 4 to 11-fold increase in absorbance values against 1.2 to 3-fold increase for both PB- and BB-LNPs, especially after 1 h reaction. These findings suggest the presence of higher amount of oxidized dimeric lignin derived products on softwood LB-LNPs, which mostly contains G-type units. When the phenoxy radicals are generated on the G-type monomers after laccase treatment, they can be shared by resonance through the 5, 1, and β position of the aromatic structure. Therefore, coupling reactions at any of these positions are favored, leading to the lignin polymerization via C—O (β-O-4, 4-O-5) and C—C type of linkages (e.g., β-β, β-5, and 5-5). However, the S-type units in the PB- and BB-LNPs display a methoxy group at the 5 position of the aromatic ring, which then prevents the formation of C—C type of linkages (e.g., β-5, and 5-5), which can explain the lower absorbance values at 515 nm for both PB- and BB-LNPs. Additionally, the results here obtained are in accordance with Py-GCMS observations, which suggested that LB-LNPs rich in G-type units is more prone to undergo polymerization reactions, while the Cα=O oxidation reaction are preferred in PB- and BB-LNPs containing S-type units.

### Evaluation of Phenolic Content

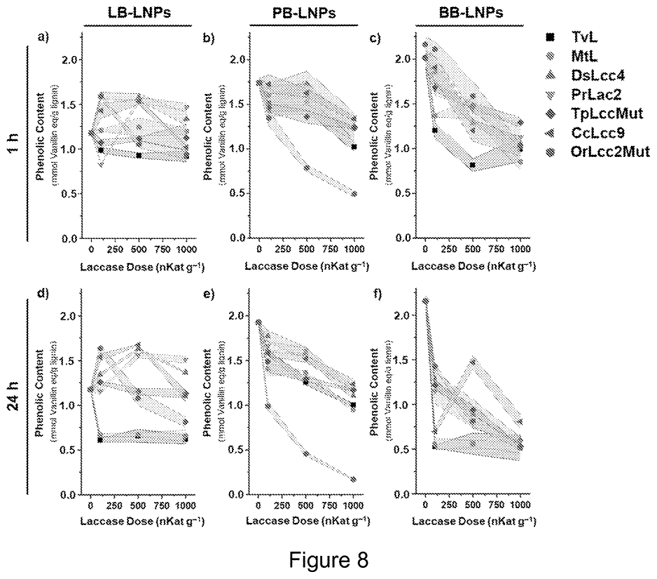

The content of phenolic hydroxyl groups can also be an indicator to understand how the laccase treatments can affect the phenolic structures on the LNP surface. Here, the phenolic content was measured using the Folin-Ciocalteu method, which is a simple, reproducible, and low-cost approach that is particularly useful when different oxidative treatments are applied to the same type of LNPs. In this way, the phenolic content was evaluated after incubating the LNP suspensions from the three different sources with the commercial and in house produced laccases, at dosages ranging from 100 to 1000 nKat g⁻¹ at pH 5 for 1 and 24 h (FIG. 8).

The phenolic hydroxyl content was similar or slightly increased after treatment of LB-LNPs with in house produced laccases (FIG. 8a, d), while for MtL and TvL was decreased, in particular after 24 h, which can indicate that LNP oxidation with the commercial laccases exhibit different kinetics. These results are in line with the abovementioned observations of the Py-GCMS and absorbance measurements on the LNPs, suggesting that the laccase-induced oxidation reaction can take place in side-chains, and the formation of dimeric products can create new phenolic hydroxyl groups displayed on the LNP surface. Conversely, the laccase-treated PB- and BB-LNPs showed an accentuated reduction of the total phenolic hydroxyl groups on their LNP surface compared to the untreated PB- and BB-LNPs (FIG. 8b, c, e, f), especially the BB-LNPs that exhibit higher percentage of S-type units in its structure. This decrease in the phenolic content was generally time- and laccase dosage-dependent, in which the PB- and BB-LNPs treated with higher laccase dosage during a longer period (24 h) presented the lowest phenolic content. This reduction in the phenolic hydroxyl levels can be ascribed to the coupling through the phenolic group, along with Cα=O oxidation reactions that are favored for S-type monomers.

### Characterization of LNPs

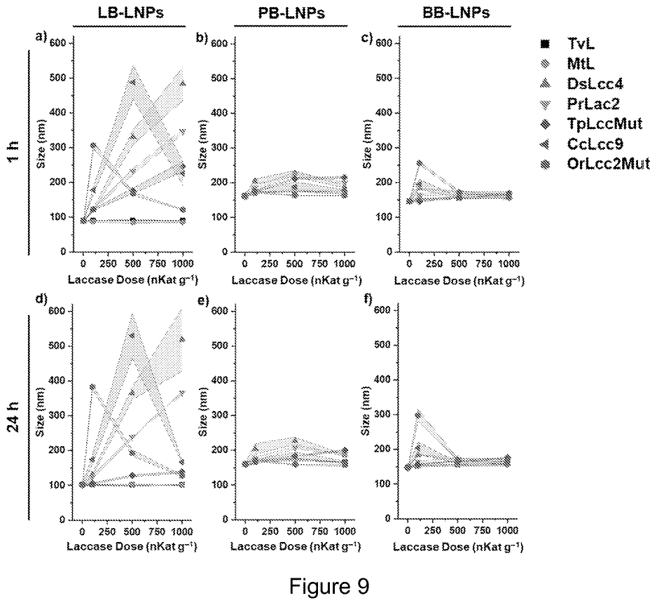

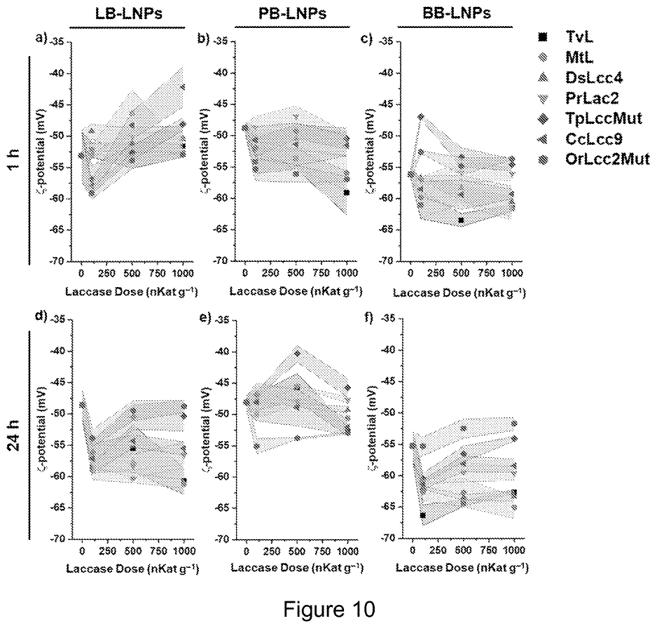

Here, the influence of the laccase treatments on the LNPs was evaluated, which were characterized for their physicochemical characteristics by determining the size (FIG. 9) and surface charge of LNPs (FIG. 10), using DLS.

As shown in FIGS. 9a, d, the LB-LNPs treated with the five in house isolated laccases experienced an accentuated increase in their particle size from about 100 up to 500 nm, while the size of LB-LNPs treated with both TvL and MtL remained similar regardless the laccase dosage and incubation time tested. In addition, the polydispersity index (PDI) of these LB-LNPs increased in the same way from ca. 0.10 up to almost 0.60, which can indicate the presence of heterogeneous LNP suspensions and formation of aggregates after laccase treatment. The fact that the in house isolated laccases led to a drastic increase in the particle size can be ascribed to the intercross-linking of particles because of the formation of oxidized dimeric products derived from the polymerization of G-type rich softwood LB-LNPs.

Contrarily to the LB-LNPs, the size of both PB- and BB-LNPs experienced a slight increase after the laccase treatments (FIG. 9b, c, e, f), along with PDI values ranging from 0.07 to 0.20 that suggest the presence of homogeneous and monodispersed LNP suspensions. The different trend on the particle size compared to the LB-LNPs can be ascribed to the preferred Cα=O oxidation reaction of PB- and BB-LNPs containing S-type units over the formation of C—C bonds. The 5-position on the aromatic ring is occupied by the methoxy group, which prevents the LNP polymerization, and therefore, the size of these LNPs exhibit a slight increase after laccase treatment.

Generally, the surface charge (ζ-potential) values of laccase-treated LNPs presented a slightly more negative charge, especially after 24 h of reaction (FIG. 10). This small change in the ζ-potential values can be due to a small increase in the number of carboxyl and Cα=O groups on the LNP surfaces, confirming the oxidation of the LNP surfaces.

### Example 3

Materials: Three lignin samples were selected for this study. Softwood kraft Lignoboost was provided by Stora Enso (Finland). Hardwood birch lignin (Betula L.) was isolated using the BLN process, and obtained from CH Bioforce Oy (Finland). Protobind 1000 was extracted from wheat straw by the soda process and acquired from GreenValue SA (Switzerland). Galactoglucomannan (GGM) and birch Glucuronoxylans (GX) were obtained from Luke (Finland). The ethanol precipitated ep Galactoglucomannan (GGM) isolated using the BLN process (epBLN-GGM) was adquired from Luke (Finland). Acetone for HPLC (299.9%), citric acid monohydratewere, and tannic acid were acquired from Sigma-Aldrich (Finland). Myceliophthora thermophila (MtL, Novozym® 51003) was purchased from Novozymes A/S (Denmark).

The heterologous expression of fungal laccases (DsLcc4, PrLac2, TpLccMut, CcLcc9, OrLcc2Mut) was performed as described in Example 1.

Preparation of LNPs: The LNPs were prepared using the acetone nanoprecipitation approach as described in Example 1.

Laccase-induced oxidation of LNPs and cross-linking with other coatings: Both Tannic acid and ethanol precipitated Galactoglucomannan isolated using the BLN process (epBLN-GGM) were previously dissolved in 25 mM citric acid pH 5 at concentration of 2, and 10 mg/mL, overnight, and further centrifuged for 10 min at 20000 g to remove the undissolved fragments. The crosslinking reactions were performed in aqueous dispersions of LB-, PB-, and BB-LNPs in 25 mM citric acid pH 5 at final concentration of 1 mg/mL, containing different concentrations of coating molecules, which were further diluted in the reaction to 1, and 2.5 mg/mL. Finally, all the laccases (MtL, DsLcc4, PrLac2, TpLccMut, CcLcc9, OrLcc2Mut) at dosage of 1000 nKat per gram (nKat/g) of lignin were added to the previous mixture, and allowed to react for 24 h. Control samples were prepared in the same way, without any coating and/or laccase treatment.

After the reaction, the samples were centrifuged at 12500 g for 15 min, washed twice with MilliQ-water, and further redispersed with MilliQ-water for further analysis.

Dynamic Light Scattering: The average hydrodynamic diameter, polydispersity index, and ζ-potential of LNPs was measured by dynamic light scattering (DLS), using a Malvern Zetasizer Nano ZS instrument (Malvern Instruments Ltd, UK). For that, the samples were diluted in MilliQ-water at a concentration of 0.5 mg/mL. Different time points (Day 1 and Week 7) were considered in order to evaluate the long term-stability of the prepared LNPs.

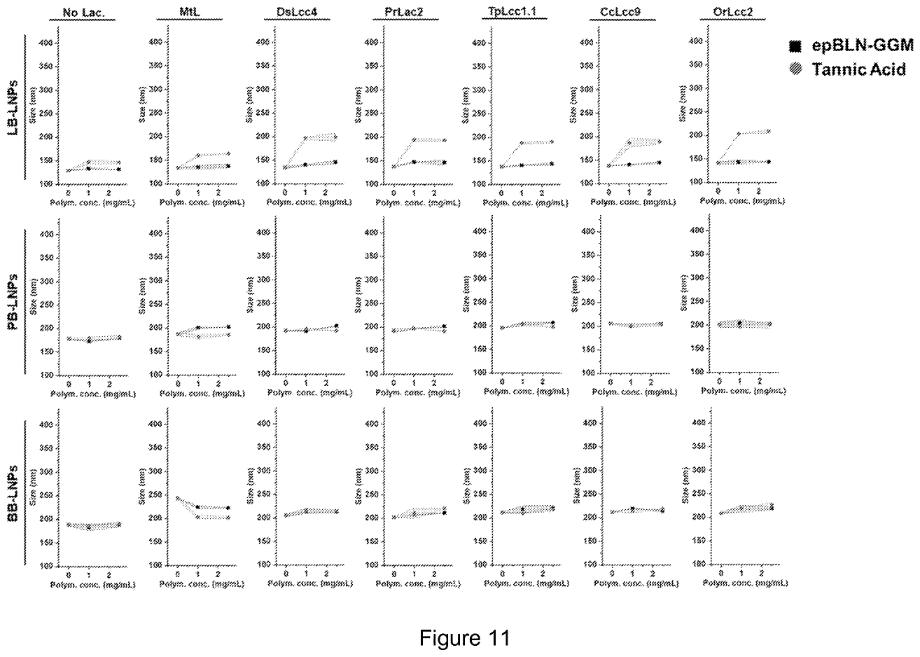

Size of polymer-coated LNPs: The size of LNPs was measured in order to evaluate the effect of the polymer coating with and without the laccase treatment (FIG. 11).

The laccase-treated softwood-derived LB-LNPs coated with tannic acid experienced an increase in particle size when compared with non-treated LNPs, as consequence of the crosslinking of lignin nanoparticles with the tannic acid polymer, possibly via the 5-position of the aromatic ring of G-type units that is free for the creation of new 5-5 bonds with the aromatic rings in the tannic acid structure, after laccase treatment. On the other hand, the PB- and BB-LNPs exhibit higher content of S-type units, which present the 5-position blocked by the methoxy group, preventing the formation of 5-5 bonds with tannic acid and consequent increase in the particle size. Therefore, their particle size did not increase as for the LB-LNPs.

Similar to the other hemicelluloses, after adding the epBLN-GGM in the reaction, the size of LNPs did not considerably change with the increase on the hemicellulose concentration, which might indicate that the hemicellulose coating can stabilize the LNP surface.

### Surface Charge of Hemicellulose-Treated LNPs:

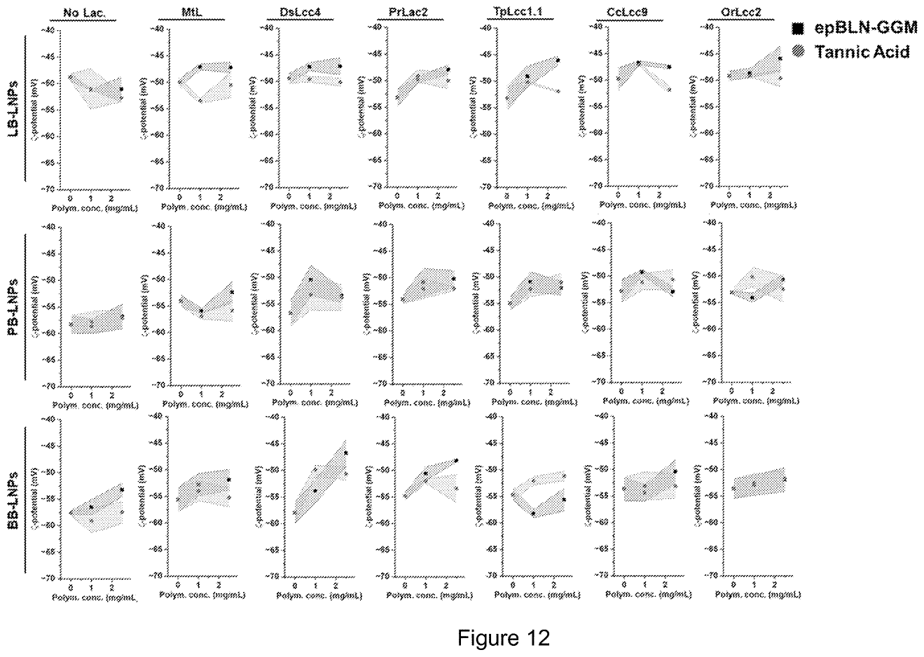

The surface charge of LNPs, here given by their ζ-potential values, was quantified in order to evaluate the effect of the concentration and type of polymer coating on the LNP coating after reaction (FIG. 12). Generally, the ζ-potential values increased as the polymer concentration in the reaction increased, which indicates that the coating was successful.

### Example 4

Hemicellulose-treated LNPs were prepared essentially as described in the previous Examples.

### Pyrolysis Gas Chromatography Mass Spectrometry (Py-GCMS)

Py-GCMS was performed as described in Example 1.

### Acid Methanolysis

Monosaccharide composition was determined by gas chromatography-flame ionization detection (GC-FID, HP 6890 N, Agilent Technologies, Waldbronn, Germany) of silylated methanolysed monosaccharide derivatives. About 10 mg of hemicellulose samples were weighed in a pear-shaped flask and suspended in 2 mL of 2 M hydrochloric acid in anhydrous methanol. The sample was incubated in an oven at 100° C. for 3 h. After cooling to room temperature 100 μL pyridine were added for neutralization and the suspension was diluted to 10 mL with methanol. An aliquot of 600 μL methanolized products were transferred to glass tubes, 100 μL methanol containing 1 mg/mL sorbitol (internal standard for neutral and acid monosaccharides determination) was added, and the sample was dried at 50° C. under a nitrogen flow. Next, 100 μL pyridine and 100 μL TMSCI/BSTFA 1:99 (v/v) were added to dry samples and silylation was performed at room temperature overnight. Silylated products were dried at 50° C. under a nitrogen flow, dissolved in 1 mL heptane, filtered through syringe filter (acrodisc, 0.45 μm) to GC vials and analyzed by GC-FID equipped with a DB-1 column (30 m, i.d. 0.25 mm, 0.25 μm film). For improving the monosaccharide quantification in lignin samples, acid methanolysis and silylation methods were optimized as follows: about 20 mg of freeze-dried lignin sample (adjusted to contain about 3 mg hemicelluloses) were used in acid methanolysis and no dilution was performed after the reaction (i.e., the 2 mL methanolized products were subjected to silylation). In silylation, the volume of 200 μL TMSCI/BSTFA 1:99 (v/v) was added to dry lignin samples instead of the 100 μL added to hemicelluloses samples to ensure complete derivatization of monosaccharides. For monosaccharide determination of hemicellulose and lignin samples, 1 mL of sample was injected and eluted at 20:1 split ratio. Temperature program consisted of keeping injected sample at 150° C. for 3 min, then increasing 2° C./min to 186° C., 1° C./min to 200° C., 20° C./min to 300° C., and holding at this temperature for 1 min. Calibration curve (with internal standard calibration) was prepared using glucose, xylose, mannose, galactose, arabinose, rhamnose, fucose, glucuronic acid, and galacturonic acid and using the highest peak of each corresponding monosaccharide. Monosaccharide determination was performed with at least two parallel replicates and the results, reported as anhydrous monosaccharide, were subjected to one-way analysis of variance (ANOVA) and Tukey test for pairwise comparison of means at 5% significance level using the Origin 2022b software.

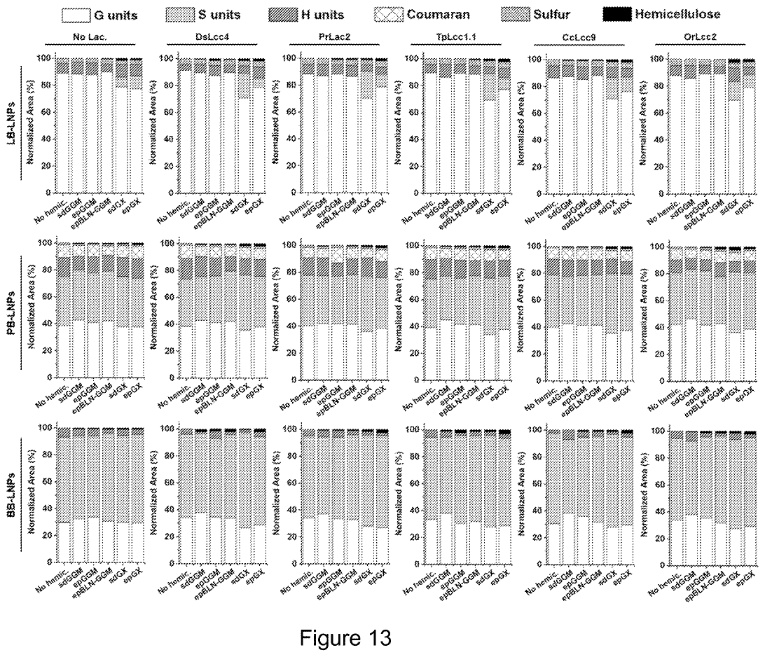

The modifications on the LNP chemical composition after hemicellulose coating were also evaluated using Py-GCMS, which allowed the analysis of LNPs by chromatographic separation and mass-spectrometric identification of the compounds released after the pyrolytic breakdown of hemicellulose-coated LNP samples, with and without laccase treatments. The peak areas were normalized for the total percentage of detected products, which were classified according to the type of lignin unit (i.e., G, H, S), and the presence of coumaran, sulfur, and hemicellulose derivatives (FIG. 13). As expected, the amount of lignin vary according to the source and processing conditions of the hemicellulose: sdGGM (28.2%), epGGM (6.6%), epBLN-GGM (34.0%), and epGX (16.6%). The lignin (1.7%), sdGX units' composition in the hemicelluloses also differ according to the hemicellulose source: softwood GGM presented mainly G units, while hardwood GX exhibited mostly S units. In addition, all hemicelluloses presented a high percentage of acetic acid. FIG. 13 shows the Py-GCMS analysis of LNPs after their incubation with the different laccases (1000 nKat/g of lignin) and hemicelluloses (2.5 mg/mL), at pH 5 and RT for 24 h. Control samples were also analyzed in the absence of laccase and/or hemicelluloses. As expected, acid acetic was the main carbohydrate-derived compound released after the pyrolytic breakdown of hemicellulose-coated LNPs. Generally, the percentage of hemicellulose-derived compounds released after the pyrolytic breakdown of hemicellulose-coated LNPs increased to a maximum of 1.2% for the hemicellulose adsorbed onto the LNPs, and 2.5% for the hemicellulose-coated LNPs assisted by laccases. For the three types of LNPs, the variation of the lignin-derived compounds released after the pyrolytic breakdown LNPs coated with ethanol precipitated hemicelluloses was not as noticeable as for the spray-dried hemicelluloses due to the lower lignin content after ethanol precipitation treatment of the spray-dried hemicelluloses.

Regarding the LB-LNPs, the percentage of G units-derived compounds after f functionalization with GGMs did not change significantly, as the LB-LNPs controls presented already high percentage of G-type compounds (>86%). On the other hand, the percentage of S units-derived compounds after coating the LNPs with S units-rich GX experienced a pronounced increase, along with the decrease of the percentage of G-type compounds. Similar to the carbohydrate-derived compounds, the sdGX-functionalized LB-LNPs treated with laccases exhibited at least double the percentage of S-type compounds than the non-treated laccase samples, which suggests that the laccase-induced crosslinking of LNPs with sdGX can happen through the lignin moieties in the GX. As for the hemicellulose-functionalized PB-LNPs, which present a similar proportion of G and S units in their composition (ca. 37% of each), the percentage of G-type compounds released after the pyrolytic breakdown of LNPs increased up to 4% when the LNPs were coated with sdGGM. In addition, the percentage of S-type compounds increased by 5% after functionalization of the LNPs with sdGX after treatment with laccases, along with the reduction in the total percentage of G-type compounds. These differences were even more pronounced in the hemicellulose-functionalized BB-LNPs, which exhibit in their composition higher percentage of S units (ca. 64%) compared to the PB-LNPs. When the sdGGM was adsorbed into the BB-LNPs, the percentage of G-type compounds increased by ca. 2%, while the laccase treatment of these samples led to an increase of 4-8%, suggesting the reaction of LNPs with the G units present in the sdGGM. In a similar way, the percentage of S-units derived compounds increased up to 8% after coating the BB-LNPs with sdGX assisted by laccases, accompanied by the reduction in the percentage of G-type compounds.

Overall, the percentage of carbohydrate-derivative compounds given by the acetic acid proportion after pyrolytic breakdown of the hemicellulose adsorbed on the LNPs, i.e. without any laccase treatment, was lower than that of the hemicellulose-coated LNPs assisted by laccases. Along with the fact that higher percentage of lignin units are present in the composition of LNPs coated with sd-hemicelluloses after treatment with laccases, the results suggest that the laccases play an active role in the crosslinking between the lignin moieties in the hemicelluloses and the LNP surface.

The analysis of the carbohydrate composition in the LNPs was carried out in order to evaluate the changes after functionalization of LNPs with hemicelluloses. First, the monosaccharide distribution in the hemicelluloses used for the reactions was assessed (data not shown). As expected, softwood-derived sd-, ep-, and epBLN-GGM exhibited mannose as their major constituent (50-64%), followed by small fractions of glucose (12.3-17.1%), galactose (5.9-12.2%) and xylose (5.8-11.0%). GGMs typically contain β-D-mannopyranosyl units and β-D-glucopyranosyl units randomly arranged and linked by 1→4 bonds, which present α-D-galactopyranosyl units linked through 1→6 bonds. The presence of xylose in softwood GGMs is due to the existence of arabinoglucuronoxylans. On the other hand, hardwood-derived sd- and ep-GX exhibited mainly xylose in their composition (>91%), derived from glucuronoxylans. Due to the difficulty in separating the lignin from hemicellulose, lignins present some carbohydrate moieties in their composition, which are covalently bound to form lignin-carbohydrate complexes. Thus, the monosaccharide composition of the technical lignins and respective LNPs was also evaluated (data not shown). It was observed that softwood lignin (LB) contained more carbohydrates than the hardwood lignin (BB), and the proportion of all carbohydrates were reduced after LNP preparation compared to the technical lignins (data not shown). This might be due to the fact that the technical lignins are initially dissolved in the acetone mixture for the LNP preparation, and the hemicelluloses are not soluble in organic solvents. After the filtration step, the acetone-insoluble fraction of lignin that can contain some hemicellulose fraction is removed, and therefore, the total percentage of carbohydrates decreased after LNP preparation from 3.1-4.3 to 1.2-2.7%. Regarding the carbohydrate composition of the technical lignins/LNPs, the differences observed in the proportion of monosaccharides are attributed to the type of LCC structures present in the lignin composition, which vary according to the lignin/hemicellulose species. Usually, the composition of LCCs extracted from softwoods involves the crosslinking of lignin with galactoglucomannans and xylans, which can explain the higher content of galactose and xylose in the LB lignin/LNPs structure (data not shown). In hardwoods, the LCC structure implies the linkage of lignin and glucuronoxylan by benzylether bonds, and therefore, the xylose was the main monosaccharide found in the BB lignin/LNPs composition (data not shown). In non-wood biomass, such as grass, arabinoxylans play an important role in LCC linkage formation with lignin moieties, which is consistent with the present results showing that xylose was the main monosaccharide in PB lignins/LNPs composition, followed by arabinose (data not shown).

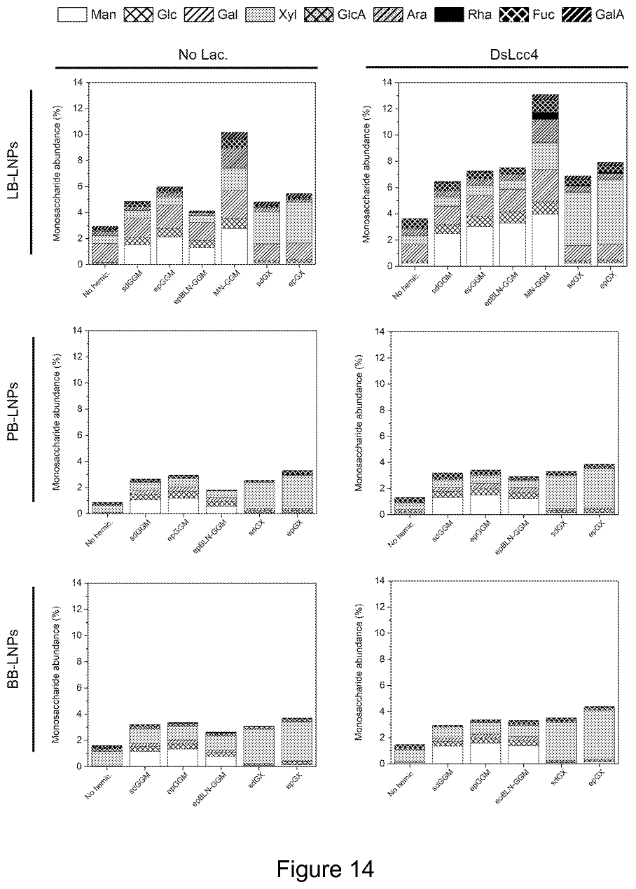

Finally, changes in the monosaccharide composition of LNPs functionalized with hemicelluloses by spontaneous absorption (no laccase) or treated with DsLcc4 as representative of all the laccases were assessed (FIG. 14).

FIG. 14 shows the monosaccharide composition of LNPs functionalized with hemicelluloses (2.5 mg/mL) by spontaneous absorption (no laccase), and treated with DsLcc4 (1000 nKat/g of lignin), at pH 5 and RT for 24 h, determined by acid methanolysis analysis. Control samples were also analyzed in the absence of hemicelluloses. MN-GGM refers to GGM obtained by a Pressurized Hot Water Extraction (PHWE) from Montinutra (Finland).

Generally, the percentage of carbohydrates in the LNP samples increased after the coating with hemicelluloses, compared to the control samples that were submitted to the same reaction conditions, but without the hemicelluloses: for LB-LNPs, the percentage of monosaccharides increased 1.4-3.6 times, while the PB- and BB-LNPs exhibited 2.1-3.8 and 1.7-3.0 times more carbohydrates, respectively. In terms of carbohydrate composition, the coating of LNPs with the softwood-derived sd-, ep- or epBLN-GGMs led to a clear increase in the percentage of mannose, which is the major component of GGMs. The percentage of mannose in the LB-LNPs increased to 1.3-2.8% after adsorption of GGMs, and it was amplified after treatment with DsLcc4 (2.5-4.0%). A similar trend was observed for both PB- and BB-LNPs, before and after laccase treatment, respectively: 0.6-1.2 vs 1.3-1.5% for PB-LNPs, and 0.8-1.3 vs 1.4-1.6% for BB-LNPs. The presence of glucose and galactose was also enlarged after functionalization of LNPs with GGMs. On the other hand, the LNPs functionalized with hardwood-derived sd- and ep-GXs exhibited an increased percentage of xylose in their composition, because of the glucuronoxylans present in the GXs structure. In addition, the percentage of xylose in GXs-coated LB-LNPs increased 4.2-5.3 times after GX adsorption, while the DsLcc4-treated LB-LNPs exhibited 6.0-7.3 times more xylose. Furthermore, the same tendency was noticed for both PB- and BB-LNPs, before and after laccase treatment, respectively: 3.8-4.8 vs 4.9-6.0 times for PB-LNPs, and 2.4-2.7 vs 3.1-4.1 times higher percentage of xylose for BB-LNPs.

Overall, the increase in mannose (GGMs) and xylose (GXs) after laccase treatment of hemicellulose-coated LNPs confirmed the results obtained with pyrolysis GCMS, where the laccase-treated LNPs functionalized with hemicelluloses exhibited higher percentage of hemicellulose-derived compounds, and the LNPs coated with sd-GGM/GX presented higher percentage of lignin units in their composition. Altogether, these results indicate that the laccase treatment can incite a more efficient crosslinking between the LNP surface and hemicelluloses.

### Example 5

Contact angles of LNPs prepared essentially as in the previous Examples were measured.

Static Contact angle measurement was performed using the Farooq et. al., method (Farooq et al., 2020, Langmuir, 36 (51), 15592-15602), with slight modification. Briefly, thin films from the prepared nanoparticles were used for spin coating on silicon wafers. Three different concentrations (0.5, 1, and 1.5 mg/ml) of LNPs were selected to double coat the silicon wafer, and the best coverage on the silicon wafer was for 1 mg/ml nanoparticles. To prepare the spin-coated specimens for contact angle measurements, silicon wafers were cut into 1.5 cm×1.5 cm square substrates, followed by plasma cleaning before spin-coating. Then, an anchoring layer of Poly-L-lysine (PLL) was applied to facilitate the physisorption of nanoparticles onto the substrate at 2000 rpm for 90 seconds, followed by nanoparticle layer deposition using the same parameters. A second deposition of nanoparticles was applied by spin coating again under the same conditions. The contact angle was measured using KSV CAM 200 Optical Contact Angle Meter. Three replications were conducted for each sample.

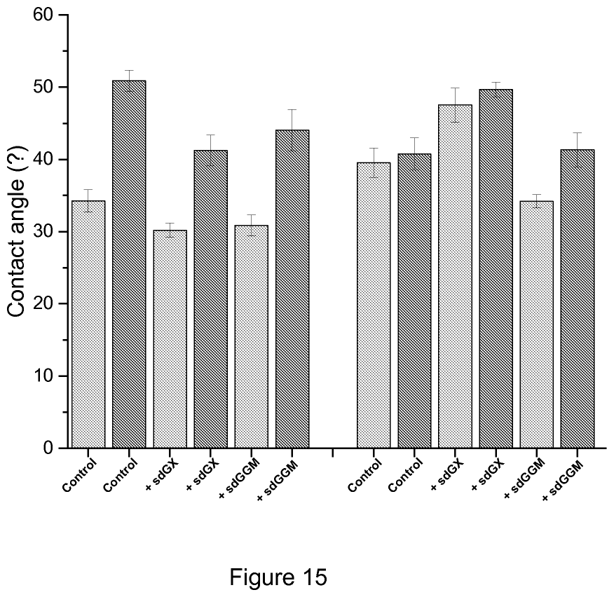

FIG. 15 shows the contact angle measurements representing the wettability (hydrophobicity and hydrophilicity) of LB-LNPs, BB-LNPs, and functionalized LNPs by hemicellulose, with and without laccase (DsLcc4) treatment. Softwood kraft Lignoboost (LB) was provided by Stora Enso (Finland), and hardwood birch lignin [Betula L. (BL)] (BLN process) was obtained from CH Bioforce Oy (Finland). Spray-dried (sd) spruce Galactoglucomannan (GGM) and birch Glucuronoxylans (GX) were obtained from Montinutra (Finland) and Luke (Finland), respectively.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A method, a product, or a use, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A method for producing lignin-hemicellulose hybrid nanoparticles, each of the lignin-hemicellulose hybrid nanoparticles comprising a lignin nanoparticle, wherein the method comprises adding hemicellulose to the surfaces of the lignin nanoparticles, thereby covering the lignin nanoparticles with hemicellulose.

2. The method according to claim 1, wherein the method comprises contacting a laccase enzyme with the hemicellulose and the lignin nanoparticles, thereby covalently crosslinking the hemicellulose to the lignin nanoparticles.

3. Lignin-hemicellulose hybrid nanoparticles, wherein each of the lignin-hemicellulose hybrid nanoparticles comprises a lignin nanoparticle and hemicellulose covering the lignin nanoparticle.

4. (canceled)

5. The method according to claim 1, wherein the ζ-potential of the lignin-hemicellulose hybrid nanoparticles in water is −42 mV or higher.

6. The method according to claim 1, wherein the average or median particle size of the lignin-hemicellulose hybrid nanoparticles is in the range of 100-500 nm.

7. The method according to claim 1, wherein the polydispersity index (PDI) of the lignin-hemicellulose hybrid nanoparticles is 0.15 or lower.

8. The method according to claim 1, wherein the lignin is technical lignin; lignin obtained from softwood, hardwood, and/or a grass; Kraft lignin, organosolv lignin, and/or lignin obtainable from an alkali/soda process, a LignoBoost process, and/or a lignosulfonate process.

9. The method according to claim 1, wherein the lignin-hemicellulose hybrid nanoparticles are stable in water or an aqueous solution for at least 4 weeks, and/or the lignin-hemicellulose hybrid nanoparticles are stable in an aqueous solution having a pH of about 3 for at least 2 hours.

10. (canceled)

11. The method according to claim 2, wherein the laccase enzyme catalyzes the crosslinking and/or polymerization of the lignin of the lignin nanoparticles at least partially, thereby increasing the average or median size of the lignin nanoparticles.

12. The method according to claim 10, wherein the laccase enzyme catalyzes the oxidation of phenolic groups of the lignin at least partially, thereby modifying the one or more properties of the lignin nanoparticles, such as one or more surface properties of the lignin nanoparticles.

13. The method according to claim 11, wherein the laccase enzyme has an amino acid sequence comprising a sequence that is at least 90% identical to at least one of the sequences set forth in SEQ ID NO: 1 (DsLcc4), SEQ ID NO: 2 (PrLac2), SEQ ID NO: 3 (TpLccMut), SEQ ID NO: 4 (CcLcc9), or SEQ ID NO: 5 (OrLcc2Mut).

14. (canceled)

15. The method according to claim 1, wherein the method further comprises grafting a functional molecule to the lignin nanoparticles.

16. (canceled)

17. The lignin-hemicellulose hybrid nanoparticles of claim 3, comprising a functional molecule grafted to the lignin nanoparticles.

18. (canceled)

19. The lignin-hemicellulose hybrid nanoparticles according to claim 3, wherein the average or median particle size of the lignin nanoparticles is in the range of 100-500 nm.

20. The lignin-hemicellulose hybrid nanoparticles according to claim 3, wherein a polydispersity index (PDI) of the lignin nanoparticles is 0.2 or lower.

21. The lignin-hemicellulose hybrid nanoparticles according to claim 3, wherein the lignin is technical lignin; lignin obtained from softwood, hardwood, and/or a grass; Kraft lignin, organosolv lignin, and/or lignin obtainable from an alkali/soda process, a LignoBoost process, and/or a lignosulfonate process.

22. The method according to claim 1, wherein the hemicellulose is spruce galactoglucomannan or birch glucuronoxylan; and/or wherein the hemicellulose is spray dried or ethanol precipitated.

23. A composition or product comprising the lignin-hemicellulose hybrid nanoparticles according to claim 3, wherein the composition or product is a pharmaceutical composition; a wound dressing; a hydrogel; a microneedle; a stabilizer; a film; a coating; a sunscreen protector; a lotion; a plastic replacement; or a food packaging product.

24. (canceled)
